(19) United States
(12) Patent Application Publication
(10) Pub. No.: US 2026/0027125 A1
(43) Pub. Date: Jan. 29, 2026

(54) AN ANTIBACTERIAL PARENTERAL FORMULATION AND METHODS THEREOF

(71) Applicant: BUGWORKS RESEARCH INDIA PVT. LTD., Bangaluru (IN)

(72) Inventors: Shahul Hameed Peer Mohamed, Bengaluru (IN); Harish Kaushik Kotakonda, Bengaluru (IN); Balasubramanian Venkataraman, Bengaluru (IN)

(21) Appl. No.: 18/994,515

(22) PCT Filed: Jul. 18, 2023
(86) PCT No.: PCT/IN2023/050697

§ 371 Date: Jan. 14, 2025

(30) Foreign Application Priority Data
Jul. 19, 2022 (IN) 202241041370

(51) Int. Cl. A61K 31/5383; A61K 9/00; A61K 9/16; A61K 9/19; A61P 31/04

(52) U.S. Cl. CPC A61K 31/5383; A61K 9/0019; A61K 9/1617; A61K 9/1623; A61K 9/1635; A61K 9/1652; A61K 9/1682; A61K 9/19; A61P 31/04

(57) ABSTRACT

The present disclosure provides a formulation comprising a compound of Formula (I) or its salt thereof with a solubilising agent, a hydrotropic agent, and a pH modifier. The present disclosure also provides a lyophilized formulation and a reconstituted formulation. The present disclosure further provides a process of preparing the formulations and methods thereof. Formula (I)

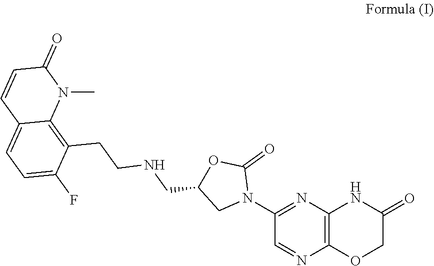 [SMILES: Cn1c(=O)ccc2ccc(F)c(CCNC[C@H]3CN(c4cnc5c(n4)NC(=O)CO5)C(=O)O3)c21]

### FIELD OF INVENTION

The present disclosure relates to the field of pharmaceutical formulations and particularly relates to antibacterial formulation. More particularly the present disclosure relates to formulations comprising a compound of Formula (I) (S)-6-(5-(((2-(7-fluoro-1-methyl-2-oxo-1,2-dihydroquinolin-8-yl) ethyl) amino) methyl)-2-oxooxazolidin-3-yl)-2H-pyrazino[2,3-b][1,4]oxazin-3(4H)-one or its salts and process of preparation of the formulations. The present disclosure also relates to lyophilized injectable pharmaceutical formulations showing improved solubility and storage stability.

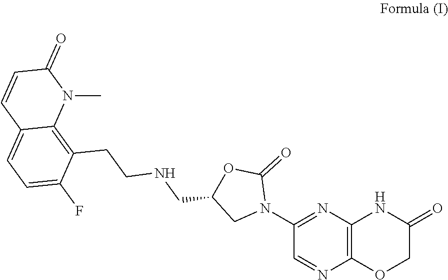 [SMILES: Cn1c(=O)ccc2ccc(F)c(CCNC[C@H]3CN(c4cnc5c(n4)NC(=O)CO5)C(=O)O3)c21]

### BACKGROUND

Globally, the ever-increasing challenge posed by the rising incidence and spread of antimicrobial resistance (AMR) has necessitated the need to amplify efforts and circumvent this major health threat looming at large. The recent pandemic has only reiterated the urgent requirement for thorough preparedness and appropriate countermeasures by the health sector to avoid large scale global catastrophes. It is essential to discover and develop novel acting, broad-spectrum antibiotics that can address AMR and offer solutions to most serious hospital associated infections. Apart from hospital and community-based health risks, the potential unexpected danger of bioterrorism is also of particular concern as it could result in major public health emergencies of severe proportions with panic, large scale disease and mortality. Hence, there is an urgent need for a safe, potent broad spectrum antibacterial agent that is effective against all the critical and high priority pathogens with multiple drug resistance background (WHO Antimicrobial resistance. https://www.who.int/news-room/fact-sheets/detail/antimicrobial-resistance). In that view a compound of Formula (I) can be identified to be suitable for developing pharmaceutical formulations towards antibiotic purpose.

Compound of Formula (I) is a fully synthetic oxazolidinone based broad spectrum antibacterial and it is chemically called as (S)-6-(5-(((2-(7-fluoro-1-methyl-2-oxo-1,2-dihydroquinolin-8-yl) ethyl) amino) methyl)-2-oxooxazolidin-3-yl)-2H-pyrazino[2,3-b][1,4]oxazin-3(4H)-one (WO2018225097). Compound of Formula (I) being as a broad-spectrum antibiotic, is a new chemical entity (NCE) with dual target inhibition and potent activity against a broad spectrum of Gram-negative bacteria including all the key members of Enterobacterales and non-fermenters such as Pseudomonas aeruginosa and Acinetobacter baumannii and Gram-positive bacteria such as Methicillin resistant Staphylococcus aureus (MRSA) and Vancomycin resistant Enterococci (VRE) (WO2018225097). Extensive MIC studies with nearly 10,000 clinical isolates, demonstrated the lack of cross-resistance to all currently used antibiotics including fluoroquinolones.

However, the compound of Formula (I) needs to be developed as parenteral antibiotic to treat various bacterial infection and to have good solubility in pharmaceutically acceptable vehicles with pH range 3-8. Further formulations or the drug product should be stable in the formulated vehicle suitable for long term storage.

Hence, clearly there remains a need to develop pharmaceutical injectable drug formulation of Formula (I) that can be prepared and stored as ready for use medicaments, and which fulfils the requirements regarding stability for longer periods of storage.

### SUMMARY OF THE INVENTION

In an aspect of the present disclosure, there is provided a formulation comprising: a) a compound of Formula (I) or its salt thereof;

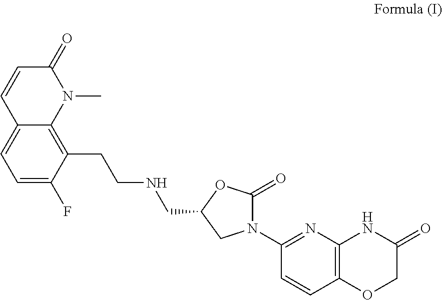 [SMILES: Cn1c(=O)ccc2ccc(F)c(CCNC[C@H]3CN(c4ccc5c(n4)NC(=O)CO5)C(=O)O3)c21]

b) a solubilising agent; c) a hydrotropic agent; and d) a pH modifier.

In another aspect of the present disclosure, there is provided a lyophilized formulation comprising: a) a compound of Formula (I) or its salt thereof;

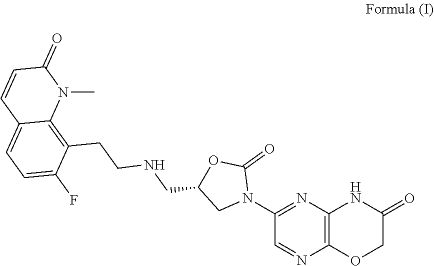 [SMILES: Cn1c(=O)ccc2ccc(F)c(CCNC[C@H]3CN(c4cnc5c(n4)NC(=O)CO5)C(=O)O3)c21]

b) a solubilising agent; c) a hydrotropic agent; and d) a pH modifier.

In one another aspect of the present disclosure, there is provided a reconstituted formulation comprising the lyophilized formulation as disclosed herein with a reconstituting agent and a diluent.

In yet another aspect of the present disclosure, there is provided a kit comprising: a) a first compartment comprising the formulation as disclosed herein; b) a second compartment comprising a facilitating agent; and c) optionally an accessory.

In further aspect of the present disclosure, there is provided a process for preparing the formulation as disclosed herein, the process comprising: a) contacting a solubilising agent with a compound of Formula (I) or it's salt in the presence of a second solvent under stirring at a temperature in a range of 18 to 40° C. to obtain a first solution; b) adding a hydrotropic agent to the first solution under stirring to obtain a second solution; and c) mixing a pH modifier to the second solution followed by addition of a vehicle to obtain the formulation.

In more aspect of the present disclosure, there is provided a process for preparing the lyophilized formulation as disclosed herein, the process comprising: a) freezing the formulation as disclosed herein, at a temperature in a range of 0 to −50° C. followed by drying at a pressure in a range of 10 pbar to 1 bar, to obtain the lyophilized formulation.

In further aspect of the present disclosure, there is provided a process for preparing the reconstituted formulation as disclosed herein, the process comprising: contacting the lyophilized formulation with a diluent followed by addition of a reconstituting agent.

In further aspect of the present disclosure, there is provided a method of treating a bacterial infection, the method comprising administering an effective amount of the formulation as disclosed herein, to a subject in need thereof.

In more aspect of the present disclosure, there is provided a method of treating or preventing a disease or a condition, the method comprising administering an effective amount of the formulation as disclosed herein, to a subject in need thereof.

These and other features, aspects, and advantages of the present subject matter will become better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF DRAWINGS

FIG. 1 illustrates XRD (x-ray diffraction) diffractogram of compound of Formula (I) formate salt, in accordance with an implementation of the present disclosure.

FIG. 2 illustrates DSC (differential scanning calorimetric) thermogram of compound of Formula (I) formate salt, in accordance with an implementation of the present disclosure.

FIG. 3 illustrates pXRD (powder x-ray diffraction) diffractogram of compound of Formula (I), in accordance with an implementation of the present disclosure.

FIG. 4 illustrates CFU/g of mouse thighs infected with Acinetobacter baumannii ATCC 17978 and treated subcutaneously with formulation of present disclosure or meropenem (200 mg/kg) or polymyxin B (25 mg/kg) administered q8h, in accordance with an implementation of the present disclosure.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “w/w” used herein refers to weight of the component with respect to total weight of the composition. The term w/v used herein refers to weight of the component with respect to total volume of the composition. Also, when water is used in the formulation then w/w and w/v shall be used interchangeably.

The term “solubilizing agent” used herein refers to a substance which assists in dissolving a substance which is otherwise poorly soluble in water. The solubilizing agent of the present disclosure includes but not limited to lactic acid, ascorbic acid, acetic acid, propionic acid, succinic acid, gluconic acid, benzoic acid, tartaric acid, glutaric acid, malic acid, and fumaric acid. The term “solubilizing agent” and “solubilizers” are used interchangeably. Solubilising agent include but are not limited to lactic acid, ascorbic acid, acetic acid, propionic acid, succinic acid, gluconic acid, benzoic acid, tartaric acid, glutaric acid, malic acid, fumaric acid or combinations thereof.

The term “hydrotropic agent” used herein refers to a chemical or a substance which is added to an incompatible mixture of two or more different substances. Hydrotropic agents are added to increase the solubility capacity of the solvent in a mixture. Hydrotropic agent includes but are not limited to niacinamide, sodium benzoate, sodium citrate, sodium acetate or combinations thereof.

The term “pH modifier” used herein refers to substances that are added to a solution or a mixture that can provide the desired pH. In the present disclosure, pH modifier includes but not limited to potassium hydroxide, sodium hydroxide, L-arginine, histidine, glycine, sodium bicarbonate or combinations thereof. The term “pH modifier” and “pH adjusting agents” are used interchangeably.

The term “vehicle” used herein refers to a substance or a liquid which is used as a medium to disperse, suspend or dissolve a material. In the present disclosure, the term “vehicle” refers to water, which is added to the formulation, to make the formulation in an injectable form and for easy use.

The term “diluent” used herein refers to a substance which is used in the to make the formulation easy to flow or pump. In the present disclosure, the diluent includes but not limited to dextrose, sorbitol, sodium bicarbonate, glucose, mannitol, sucrose, or sodium chloride.

The term “reconstituting agent” used herein refers to a substance which is added to a dry substance and converts the dry substance in a fluid form. In the present disclosure, the reconstituting agent refers to a substance which is added to a lyophilized formulation and makes the formulation easy for use, preferably as an injection formulation. The reconsitituting agent of the present disclosure includes but is not limited to water. The lyophilized formulation is added with reconstituting agent to obtain the reconstituted formulation.

The term “lyophilized formulation” used herein refers to a dried formulation wherein the formulation prepared is subjected to sequential process such as heating and freezing to obtain the lyophilized formulation. The lyophilized formulation enhances the stability of the active component in the formulation and provides a long term storage of the drug product. The lyophilized formulation is also referred to as lyophilized cake, wherein the as-prepared formulation is subjected to processes selected from heating, freezing, drying or combinations thereof, which are carried out in a sequential manner, to obtain the lyophilized formulation. The process used in obtaining the lyophilized formulation is also called as lyocycle.

The term “therapeutic agent” used herein refers to a substance or a pharmaceutical material which is capable of imparting therapeutic effect such as diagnosis, prevention, curing, alleviate, or treat a disease, a disorder, a condition or an infection.

A term once described, the same meaning applies for it, throughout the disclosure.

As discussed in the background, the compound of Formula (I) has the potential to treat various bacterial infections. Especially the compound of Formula (I) is capable of treating a variety of clinical indications caused by both Gram-positive and Gram-negative bacterial species, such as complicated and uncomplicated urinary tract infections (cUTI, eg., pyelonephritis, cystitis), intra-abdominal infections (cIAI), bloodstream infections, Hospital-Acquired Bacterial Pneumonia (HABP, Nosocomial Pneumonia) and Ventilator-Associated Bacterial Pneumonia (VABP), Community-Acquired Bacterial Pneumonia (CABP), cystic fibrosis secondary infections (CFI), skin and soft tissue infections (SSTIs), endocarditis, meningitis, dysentery and diarrhoae, typhoid, Clostridium difficile associated colitis and diarrhoea (CDAD), Helicobacter pylori associated peptic ulcer. It also has the potential to treat bacterial sexually transmitted diseases caused by Chlamydia trachomatis, gonorrhoea caused by Neisseria gonorrhoeae and syphilis caused by Treponema pallidum as well as bioterrorism associated bacterial diseases caused by Anthrax (Bacillus anthracis), Bubonic Plague (Yersinia pestis), Tularemia (Francisella tularensis), Glanders (Burkholderia mallei), Melioidosis (Burkholderia pseudomallei) and Q fever (Coxiella burnetii).

However, the solubility of the compound of Formula (I) or its salts thereof, is restricted and therefore the bioavailability may be limited. It is to be noted that the compound of Formula (I) contains an amine group and therefore it can easily form salts with acids including the hydrochloride, formate, acetate etc. But then, the salt of form of compound of Formula (I) has very limited solubility in water (2-4 mg/ml) and requires further solubility improvement in 20-50 mg/ml strength in a suitable pharmaceutical vehicle with the pH compatible for parenteral route administration for human use. Accordingly, the present disclosure provides a formulation comprising the compound of Formula (I) with various components such as solubilising agent, hydrotropic agent and pH modifier. Also, the formulation needs to be stable for long duration and the stability of the injection form of the formulation should also be maintained. The formulation of the present disclosure uses combination of acids for better solubility and further a pH modifier to balance the pH caused due to the combination of acid. Further, the formulation of the present disclosure uses a suitable hydrotropic agent to maintain maximum solubility of the compound of Formula (I) in the formulation. In addition, the present disclosure provides a lyophilized formulation, a reconstituted formulation, and processes for preparing the same.

In an embodiment of the present disclosure, there is provided a formulation comprising: a) a compound of Formula (I) or its salt thereof;

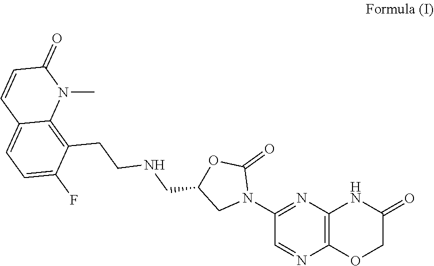 [SMILES: Cn1c(=O)ccc2ccc(F)c(CCNC[C@H]3CN(c4cnc5c(n4)NC(=O)CO5)C(=O)O3)c21]

b) a solubilising agent; c) a hydrotropic agent; and d) a pH modifier.

In an embodiment of the present disclosure, there is provided a formulation as disclosed herein, wherein the formulation comprises the compound of Formula (I) or its salt thereof, in a weight range of 1 to 30% (w/w); the solubilising agent in a weight range of 2 to 55% (w/w); the hydrotropic agent in a weight range of 1 to 30% (w/w); and the pH modifier in a weight percentage range of 0.5 to 30% (w/w).

In another embodiment of the present disclosure, there is provided a formulation as disclosed herein, wherein the formulation comprises the compound of Formula (I) or its salt thereof, in a weight range of 2 to 20% (w/w); the solubilising agent in a weight range of 5 to 35% (w/w); the hydrotropic agent in a weight range of 1 to 20% (w/w); and the pH modifier in a weight percentage range of 0.5 to 20% (w/w).

In an embodiment of the present disclosure, there is provided a formulation as disclosed herein, wherein the solubilising agent is selected from lactic acid, ascorbic acid, acetic acid, propionic acid, succinic acid, gluconic acid, benzoic acid, tartaric acid, glutaric acid, malic acid, fumaric acid, or combinations thereof; the pH modifier is selected from potassium hydroxide, sodium hydroxide, 1-arginine, histidine, glycine, sodium bicarbonate, or combinations thereof; the hydrotropic agent is selected from niacinamide, sodium benzoate, sodium citrate, or sodium acetate; and the salt of compound of Formula (I) is selected from formate, mesylate, esylate, besylate, tosylate, actetate, propionate, fumarate, maleate, tartarate, succinate, glycolate, glutamate, aspartate, hydrochloride, hydrobromide, or sulphate. In another embodiment of the present disclosure, wherein the solubilising agent is selected from lactic acid, ascorbic acid, acetic acid, or combinations thereof; the pH modifier is selected from potassium hydroxide, 1-arginine, or combinations thereof; and the hydrotropic agent is niacinamide, or sodium benzoate.

In an embodiment of the present disclosure, there is provided a formulation as disclosed herein, wherein the solubilising agent is a combination of lactic acid, and ascorbic acid; the pH modifier is 1-arginine; the hydrotropic agent is niacinamide; and the salt of compound of Formula (I) is formate.

In an embodiment of the present disclosure, there is provided a formulation as disclosed herein, wherein the formulation is stable up to a temperature in a range of −70° C. to 10° C.

In an embodiment of the present disclosure, there is provided a formulation as disclosed herein, wherein the formulation has a pH in a range of 2 to 6. In another embodiment of the present disclosure, the formulation has a pH in a range of 3 to 5.

In an embodiment of the present disclosure, there is provided a formulation as disclosed herein, wherein the formulation comprises a vehicle; and the vehicle is water.

In an embodiment of the present disclosure, there is provided a formulation as disclosed herein, wherein the formulation is capable of killing or inhibiting the growth of microorganisms, and the microorganism is selected from bacteria, virus, fungi, or protozoa.

In an embodiment of the present disclosure, there is provided a formulation as disclosed herein, wherein the formulation is an anti-infective agent against microorganisms, in particular bacteria; and the bacteria is selected from gram negative bacteria, gram positive bacteria, or combinations thereof.

In an embodiment of the present disclosure, there is provided a formulation as disclosed herein, wherein the microorganism is selected from Escherichia coli, Pseudomonas aeruginosa, Klebsiella pneumoniae, Acinetobacter baumannii, Enterobacter cloacae, Citrobacter spp., Proteus spp., Serratia marcescens, Salmonella species, Morganella morganii, Klebsiella oxytoca, Klebsiella aerogenes, Providencia spp., Neisseria gonorrhoea, Mycoplasma spp., Campylobacter sp, Fusobacterium spp., Bacteroides fragilis, Prevotella sp., Shigella sp., Helicobacter pylori, Ureaplasma spp., Burkholderia gladioli, Burkholderia multivorans, Pandorea apista, Burkholderia cepacia species, Burkholderia pseudomallei, Burkholderia mallei, Stenotrophomonas maltophilia, Achromobacter spp., Ralstonia picketii, Legionella pneumophila, Clostridium difficile, Staphylococcus aureus, Coagulase-negatives Staphylococcus, Streptococcus pyogenes, Streptococcus pneumoniae, Enterococcus faecium, Enterococcus faecalis, Bacillus anthracis, Yersinia pestis and Francisella tularensis.

In an embodiment of the present disclosure, there is provided a formulation as disclosed herein, wherein the formulation inhibits an enzyme selected from bacterial gyrase, topoisomerase IV, or combinations thereof.

In an embodiment of the present disclosure, there is provided a formulation as disclosed herein, wherein the formulation further comprises an additive, a therapeutic agent, or combinations thereof.

In an embodiment of the present disclosure, there is provided a formulation as disclosed herein, wherein the additive is selected from sorbitol, dextrose, sodium phosphate monobasic, sodium phosphate dibasic, sodium citrate, sodium bicarbonate, sodium chloride, or combinations thereof.

In an embodiment of the present disclosure, there is provided a formulation comprising: a) a compound of Formula (I) or its salt thereof; b) a solubilising agent; c) a hydrotropic agent; d) a pH modifier; and e) a vehicle.

In an embodiment of the present disclosure, there is provided a formulation comprising: a) a compound of Formula (I) or its salt thereof; b) a solubilising agent; c) a hydrotropic agent; d) a pH modifier; e) a vehicle; and f) an additive.

In an embodiment of the present disclosure, there is provided a formulation comprising: a) a compound of Formula (I) or its salt thereof; b) a solubilising agent; c) a hydrotropic agent; d) a pH modifier; e) a vehicle; f) an additive; and g) a therapeutic agent.

In an embodiment of the present disclosure, there is provided a formulation comprising: a) a compound of Formula (I) or its salt thereof; b) a solubilising agent selected from lactic acid, ascorbic acid, acetic acid, propionic acid, succinic acid, gluconic acid, benzoic acid, tartaric acid, glutaric acid, malic acid, fumaric acid or combinations thereof; c) a hydrotropic agent selected from niacinamide, sodium benzoate, sodium citrate and sodium acetate; and d) a pH modifier selected from potassium hydroxide, sodium hydroxide, 1-arginine, histidine, glycine, sodium bicarbonate, or combinations thereof.

In an embodiment of the present disclosure, there is provided a formulation comprising: a) a compound of Formula (I) or its salt thereof; b) a solubilising agent selected from lactic acid, ascorbic acid, acetic acid, propionic acid, succinic acid, gluconic acid, benzoic acid, tartaric acid, glutaric acid, malic acid, fumaric acid or combinations thereof; c) a hydrotropic agent selected from niacinamide, sodium benzoate, sodium citrate and sodium acetate; d) a pH modifier selected from potassium hydroxide, sodium hydroxide, l-arginine, histidine, glycine, sodium bicarbonate, or combinations thereof; and e) water as vehicle.

In an embodiment of the present disclosure, there is provided a formulation comprising: a) a compound of Formula (I) formate salt; b) a solubilizing which is a combination of ascorbic acid and lactic acid; c) niacinamide; and d) l-arginine.

In an embodiment of the present disclosure, there is provided a lyophilized formulation comprising: a) a compound of Formula (I) or its salt thereof; b) a solubilising agent; c) a hydrotropic agent; and d) a pH modifier.

In an embodiment of the present disclosure, there is provided a reconstituted formulation comprising: the lyophilized formulation comprising: a) a compound of Formula (I) or its salt thereof; b) a solubilising agent; c) a hydrotropic agent; and d) a pH modifier, with a reconstituting agent and a diluent.

In an embodiment of the present disclosure, there is provided a reconstituted formulation as disclosed herein, the reconstituting agent is water; the diluent is selected from dextrose, sorbitol, sodium bicarbonate, glucose, mannitol, sucrose, or sodium chloride; and the diluent is in a weight range of 0.9-25% (w/v) in a first solvent. In another embodiment, the diluent is in a weight range of the diluent is in a weight range of 1-10% (w/v) in a first solvent. In one another embodiment, the diluent is in a weight range of the diluent is in a weight range of 2-10% (w/v) in a first solvent.

In an embodiment of the present disclosure, there is provided a reconstituted formulation as disclosed herein, the first solvent is water.

In an embodiment of the present disclosure, there is provided a reconstituted formulation comprising: i) the lyophilized formulation comprising: a) a compound of Formula (I) or its salt thereof; b) a solubilising agent; c) a hydrotropic agent; d) a pH modifier; ii) a reconstituting agent; and iii) a diluent.

In an embodiment of the present disclosure, there is provided a reconstituted formulation comprising: a) a compound of Formula (I) or its salt thereof; b) a solubilising agent selected from lactic acid, ascorbic acid, acetic acid, propionic acid, succinic acid, gluconic acid, benzoic acid, tartaric acid, glutaric acid, malic acid, fumaric acid or combinations thereof; c) a hydrotropic agent selected from niacinamide, sodium benzoate, sodium citrate and sodium acetate; d) a pH modifier selected from potassium hydroxide, 1-arginine, histidine, glycine, sodium bicarbonate, or combinations thereof; e) water as reconstituting agent; and f) a diluent selected from dextrose, sorbitol, sodium bicarbonate, glucose, mannitol, sucrose, or sodium chloride.

In an embodiment of the present disclosure, there is provided a reconstituted formulation comprising: a) a compound of Formula (I) formate salt; b) a solubilizing agent which is a combination of ascorbic acid and lactic acid; c) niacinamide; d) 1-arginine; e) water as reconstituting agent; and f) dextrose as a diluent.

In an embodiment of the present disclosure, there is provided a kit comprising: a) a first compartment comprising the formulation as disclosed herein; b) a second compartment comprising a facilitating agent; and c) optionally an accessory.

In an embodiment of the present disclosure, there is provided a kit comprising: a) a first compartment comprising the formulation as disclosed herein; b) a second compartment comprising water for injection; and c) optionally an accessory selected from diluent bag, infusion tubing, needles, connector, or combinations thereof.

In an embodiment of the present disclosure, there is provided a process for preparing the formulation, the process comprising: a) contacting a solubilising agent with a compound of Formula (I) or its salt in the presence of a second solvent under stirring at a temperature in a range of 18 to 40° C. to obtain a first solution; b) adding a hydrotropic agent to the first solution under stirring to obtain a second solution; and c) mixing a pH modifier to the second solution followed by addition of a vehicle to obtain the formulation.

In an embodiment of the present disclosure, there is provided a process for preparing the formulation as disclosed herein, wherein the solubilising agent is selected from ascorbic acid, lactic acid, acetic acid, succinic acid, or combinations thereof; and two or more solubilising agents are mixed prior to contacting with the compound of Formula (I).

In an embodiment of the present disclosure, there is provided a process for preparing the formulation as disclosed herein, wherein the first solution has a pH in a range of 2 to 3; and the second solution has a pH in a range of 2.5 to 4.

In an embodiment of the present disclosure, there is provided a process for preparing the formulation as disclosed herein, wherein the formulation is filtered after adding the vehicle.

In an embodiment of the present disclosure, there is provided a process for preparing the formulation as disclosed herein, wherein the second solvent is water; and the vehicle is water.

In an embodiment of the present disclosure, there is provided a process for preparing the lyophilized formulation, the process comprising: a) freezing the formulation as disclosed herein at a temperature in a range of 0 to −50° C. followed by drying at a pressure in a range of 10 pbar to 1 bar, to obtain the lyophilized formulation.

In an embodiment of the present disclosure, there is provided a process for preparing the lyophilized formulation as disclosed herein, wherein freezing is carried out for a time period in a range of 30 minutes to 30 hours; and drying is carried out for a time period in a range of 20 minutes to 150 hours. In another embodiment of the present disclosure, freezing is carried out for a time period in a range of 1 hour to 25 hours; and drying is carried out for a time period in a range of 1 hour to 140 hours. In one another embodiment of the present disclosure, wherein freezing is carried out for a time period in a range of 10 hours to 25 hours; and drying is carried out for a time period in a range of 50 hours to 130 hours.

In an embodiment of the present disclosure, there is provided a process for preparing the reconstituted formulation, the process comprising: contacting the lyophilized formulation with a diluent followed by addition of a reconstituting agent.

In an embodiment of the present disclosure, there is provided a process for preparing the reconstituted formulation as disclosed herein, wherein the process is carried out for a time period in a range of 60 seconds to 10 minutes.

In an embodiment of the present disclosure, there is provided a process for preparing the reconstituted formulation as disclosed herein, wherein the reconstituted formulation is stable for a time period range of 20 to 30 hours at a temperature in a range of 20 to 35° C.

In an embodiment of the present disclosure, there is provided a process for preparing the reconstituted formulation as disclosed herein, wherein the reconstituted formulation has a pH in a range of 3 to 5; and has osmolality in a range of 300-500 mOs-mol/kg.

In an embodiment of the present disclosure, there is provided use of the formulation or the lyophilized formulation or the reconstituted formulation as disclosed herein.

In an embodiment of the present disclosure, there is provided use of the kit as disclosed herein.

In an embodiment of the present disclosure, there is provided use of the formulation or the lyophilized formulation or the reconstituted formulation as disclosed herein, for manufacture of a medicament.

In an embodiment of the present disclosure, there is provided a method of treating a bacterial infection, the method comprising administering an effective amount of the formulation as disclosed herein to a subject in need thereof.

In an embodiment of the present disclosure, there is provided a method of treating a bacterial infection, the method comprising administering an effective amount of the lyophilized formulation as disclosed herein to a subject in need thereof.

In an embodiment of the present disclosure, there is provided a method of treating a bacterial infection, the method comprising administering an effective amount of the reconstituted formulation as disclosed herein to a subject in need thereof.

In an embodiment of the present disclosure, there is provided a method of treating a bacterial infection as disclosed herein, wherein the bacterial infection is caused by bacteria, virus, fungi, or protozoa.

In an embodiment of the present disclosure, there is provided a method of treating or preventing a disease or a condition, the method comprising administering an effective amount of the formulation as disclosed herein to a subject in need thereof.

In an embodiment of the present disclosure, there is provided a method of treating or preventing a disease or a condition, the method comprising administering an effective amount of the lyophilized formulation as disclosed herein to a subject in need thereof.

In an embodiment of the present disclosure, there is provided a method of treating or preventing a disease or a condition, the method comprising administering an effective amount of the reconstituted formulation as disclosed herein to a subject in need thereof.

In an embodiment of the present disclosure, there is provided a method of treating or preventing a disease or a condition as disclosed herein, wherein the disease or the condition is mediated by gram-positive, gram-negative bacterial species or combinations thereof.

In an embodiment of the present disclosure, there is provided a method of treating or preventing a disease or a condition as disclosed herein, wherein the disease or the condition is selected from complicated and uncomplicated urinary tract infections (cUTI, eg., pyelonephritis, cystitis), intra-abdominal infections (cIAI), bloodstream infections, Hospital-Acquired Bacterial Pneumonia (HABP, Nosocomial Pneumonia), Ventilator-Associated Bacterial Pneumonia (VABP), Community-Acquired Bacterial Pneumonia (CABP), cystic fibrosis secondary infections (CFI), skin and soft tissue infections (SSTIs), endocarditis, meningitis, dysentery and diarrhoae, typhoid, Clostridium difficile associated colitis and diarrhoea (CDAD), Helicobacter pylori associated peptic ulcer, sexually transmitted diseases caused by Chlamydia trachomatis, gonorrhoea caused by Neisseria gonorrhoeae, syphilis caused by Treponema pallidum, or bioterrorism associated bacterial diseases caused by Anthrax (Bacillus anthracis), Bubonic Plague (Yersinia pestis), Tularemia (Francisella tularensis), Glanders (Burkholderia mallei), Melioidosis (Burkholderia pseudomallei) and Q fever (Coxiella burnetii).

Although the subject matter has been described in considerable detail with reference to certain embodiments thereof, other embodiments are possible. As such, the spirit and scope of the disclosure should not be limited to the description of the embodiments contained herein.

### EXAMPLES

The following examples provide the details about the synthesis, activities and applications of the compounds and formulations of the present disclosure. It should be understood the following is representative only, and that the disclosure is not limited by the details set forth in these examples.

### Abbreviations

XRD—X-ray Diffraction
DMSO—Dimethyl sulfoxide
CFU—colony forming unit
MIC—Minimum inhibitory concentration
pXRD—powder X-ray Diffraction
PPt—precipitate
PBS—Phosphate buffer solution
NMT—Not more than
WFI—Water for Injection
mL—MilliLiter
mg—Milligram
g—Gram
Qs—Quantity Sufficient
° C.—Degree centigrade
Hrs—Hours
RH—Relative Humidity
FD—Formulation development
API—Active pharmaceutical Ingredient
Ppt—Precipitation
PVP K-12—Polyvinyl Pyrrolidine
HPP-CD—Hydroxy Propyl Peta Cyclodextrin
NaOH—Sodium Hydroxide
N—Normality
RT—Room Temperature
USP—United State Pharmacopeia
Min—Minute
mT—milliTorr
R/H—Ramp/Hold
IV—Intravenous
w/w—weight/weight
Sec—Seconds
mM—milliMoles

### Methods and Materials

For the purpose of the present disclosure the following materials were purchased and were used without further purification.

| | Name of | Pharma- | | |
|---|---|---|---|---|
| Sr. | components | copoeial | | |
| No. | used | Grade | CAS No. | Manufacturer |
| 1 | Ascorbic acid | Ph. Eur./USP | 50-81-7 | CG Chemikalien |
| | | | | GmbH & Co. KG |
| 2 | Lactic acid | USP/Ph. Eur | 79-33-4 | Finar Ltd |
| 3 | Niacinamide | USP | 98-92-0 | Amsal Chem Pvt |
| | | | | Ltd |
| 5 | L-Arginine | USP/JP/EP/BP | 74-79-3 | JT Baker |
| | | | | (Avantor) |
| 6 | WFI | — | — | In-house |
| 7 | Glacial Acetic | USP | 64-19-7 | Sigma-Aldrich |
| | Acid | | | (Merck |
| | | | | Millipore) |
| 8 | Hydroxypropyl | USP/EP | 128446-35-5 | Sigma-Aldrich |
| | β-CD | | | (Merck |
| | | | | Millipore) |
| 9 | Polyvinyl | — | 9003-39-8 | TCI Chemicals |
| | Pyrrolidine K- | | | India PVT Ltd |
| | 12 | | | |
| 10 | Succinic acid | USP | 110-15-6 | Sigma-Aldrich |
| | | | | (Merck |
| | | | | Millipore)/TCI |
| | | | | Chemicals |
| 11 | Citric acid | (Ph. Eur.) | 77-92-9 | Merck Millipore |
| 12 | Dextrose | Ph Eur, BP, | 77938-63-7 | Merck Millipore |
| | | JP, USP | | |
| 13 | Glucose | (USP, BP, Ph. | 50-99-7 | HIMEDIA/Merck |
| | | Eur.) | | Millipore |

Powder Xray diffraction analysis was carried out using Instrument from Bruker model number D2 PHASER. Thermal analysis and the differential scanning calorimetry was determined using Perkin Elmer Instrument Model No: DSC8000.

### Example 1

### Compound of Formula (I)

The compound of Formula (I) was prepared by the process defined in WO20188225097 and was obtained as crystalline formate salt (S)-6-(5-(((2-(7-fluoro-1-methyl-2-oxo-1,2-dihydroquinolin-8-yl) ethyl) amino) methyl)-2-oxooxazolidin-3-yl)-2H-pyrazino[2,3-b][1,4]oxazin-3(4H)-one formate with single polymorph characteristics.

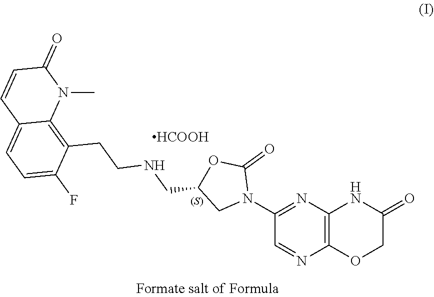

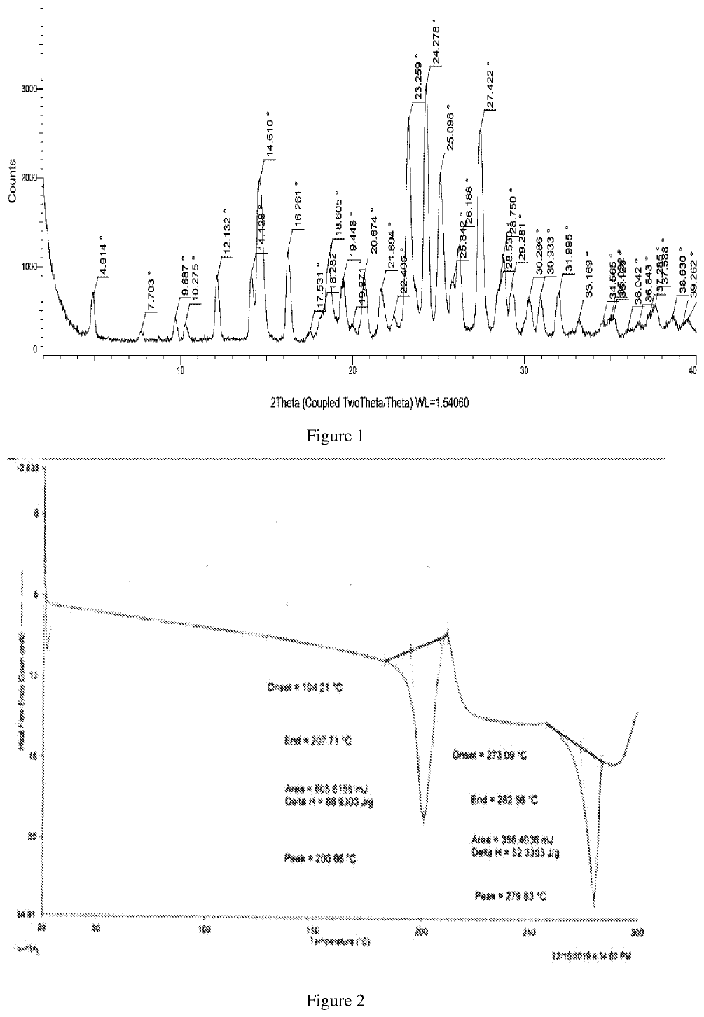

The prepared formate salt of the compound of Formula (I) was subjected to powder x-ray diffraction analysis and the characteristic single polymorphic XRD diffractogram is shown in FIG. 1. The well defined pattern of the diffractoram confirmed that a single polymorph was formed with crystalline characteristics. FIG. 2 depicts the differential scanning calorimetric (DSC) thermogram of the formate salt of the compound of Formula (I). It could be inferred that the compound of Formula (I) is crystalline in nature as indicated by high and sharp peak of the thermogram.

### Example 2

### Antibacterial Activity Determination

The compound of Formula (I) exhibit very potent antibacterial activity against various bacterial species which includes Gram-negative and Gram-positive, aerobes and anaerobes and biothreat bacterial pathogens. The bacterial susceptibility testing procedures for various bacterial species described herein.

### MIC Broth Microdilution Methods

The following MIC broth microdilution method was used for all bacterial strains other than Neisseria gonorrhoea, Helicobacter pylori and the anaerobes:

Antimicrobials were prepared in line with CLSI susceptibility testing standards (Clinical and Laboratory Standards Institute CLSI. 2018. Methods for Dilution Antimicrobial Susceptibility Tests for Bacteria That Grow Aerobically. 11th ed. CLSI standard M07). Solutions of antimicrobials or combinations at 2×the final concentrations were prepared in 50 mL tubes by dilution in CA-MHB (or Buffered yeast extract alpha ketoglutarate Broth for Legionella pneumophila) and transferred manually into 96-well master blocks. 50 L of each well were then transferred from the master block into 96-well plates. Microtiter plates were stored frozen at −80° C. until the day of test for a maximum of 6 months and never freeze/thawed more than once. Bacterial inocula were prepared at approximately 1×10⁶ CFU/mL by diluting 100-fold a 0.5 McFarland suspension in CA-MHB with TES. Antibacterial panel wells were thawed at room temperature and then 50 μL of inoculum added to each well to give a final density of approximately 5×10⁵ CFU/mL and desired test concentrations of antibacterial agents. Test plates were incubated according to CLSI guidelines and read visually. MIC values corresponded to the first well with no visible growth.

Agar Dilution method for Neisseria gonorrhoea/Helicobacter Pylori

Compound of Formula (I) was dissolved at 3200 g/mL in DMSO. Solutions at 100×the concentrations of the test ranges were made in DMSO by serial dilutions and then diluted 100-fold in GC agar media (for Neisseria gonorrhoea) or Mueller-Hinton agar supplemented with 5.0% aged (≥2-week-old) sheep blood (for H. pylori) and poured into dishes (final volume=40 mL). MIC tests were performed by agar dilution in line with CLSI susceptibility testing standards (CLSI. 2021. Performance Standards for Antimicrobial Susceptibility Testing. 31st ed. CLSI supplement M100). Bacterial inocula were prepared by a direct colony suspension method. Bacterial suspensions were adjusted to a 0.5 McFarland in 0.9% NaCl. 200 μL of the bacterial suspensions were placed into wells of a 96-well plate, and 1 μL of each suspension (corresponding to approximately 1×10⁴ CFU) was deposited onto the agar surface. The plates were incubated according to CLSI guidelines (CLSI. 2018. Methods for Dilution Antimicrobial Susceptibility Tests for Bacteria That Grow Aerobically. 11th ed. CLSI standard M07).

### Agar Dilution Method for Antimicrobial Testing of Anaerobes (Bacteroides Species, Fusobacterium Species, Prevotella Species and Clostridium Species)

Compound of Formula (I) or its salt preferably the formate salt of the compound of Formula (I) was dissolved at 3200 g/mL in DMSO. Solutions at 100× the concentrations of the test ranges were made in DMSO by serial dilutions and then diluted 100-fold in Brucella agar supplemented with 5 g/mL hemin, 1 g/mL vitamin K1, and 5.0% laked sheep blood. media and poured into dishes (final volume=40 mL). The solutions were incubated for 42-48 hours at 35-37° C. using a Bactron 600 Anaerobic Chamber containing an atmosphere of 5% CO₂, 5% H₂, and 90% N₂ prior to determining the MIC values (CLSI. 2021. Performance Standards for Antimicrobial Susceptibility Testing. 31st ed. CLSI supplement M100).

Minimum Inhibitory Concentration (MIC) is the lowest concentration of the active component or the drug which facilitates arresting of the bacterial growth. The MIC₅₀ (Minimum Inhibitory Concentration arresting the bacteria growth of 50% of species tested) and MIC₉₀ (Minimum Inhibitory Concentration arresting the bacteria growth of 90% of species tested) were determined and is tabulated in Table 1 below. The formate salt of the compound of Formula (I) demonstrated potent antibacterial activity against Gram positive, Gram negative, anaerobes, and biothreat bacterial pathogens (Table 2).

TABLE 1
MIC₉₀ results against clinical isolates of Gram-negative and Gram-positive
bacterial species for compound of Formula (I) formate salt

| | | MIC Range | | MIC₅₀ | MIC₉₀ |
|---|---|---|---|---|---|
| | N | (μg/ml) | | (μg/ml) | (μg/ml) |
| GRAM-NEGATIVE | | | | | |
| BACTERIA | | | | | |
| Escherichia coli | 1128 | ≤0.015 | 4 | 0.12 | 0.5 |
| Pseudomonas aeruginosa | 985 | <0.015 | 8 | 0.5 | 1 |
| Klebsiella pneumoniae | 945 | 0.03 | 32 | 0.5 | 2 |
| Acinetobacter baumannii | 860 | 0.03 | 4 | 0.25 | 0.5 |
| Enterobacter cloacae | 604 | ≤0.015 | 32 | 0.25 | 1 |
| Citrobacter freundii | 390 | 0.03 | 8 | 0.25 | 1 |
| Proteus mirabilis, | 384 | 0.03 | 2 | 0.12 | 0.5 |
| vulgaris | | | | | |
| Serratia marcescens | 367 | 0.03 | 32 | 0.25 | 1 |
| Stenotrophomonas | 290 | 0.03 | 2 | 0.06 | 0.25 |
| maltophilia | | | | | |
| Salmonella species | 184 | 0.03 | 2 | 0.12 | 0.25 |
| Morganella morgannii | 165 | ≤0.015 | 8 | 0.12 | 0.5 |
| Klebsiella oxytoca, | 137 | 0.06 | 4 | 0.25 | 0.5 |
| aerogenes | | | | | |
| Neisseria gonorrhoea | 128 | ≤0.015 | ≤0.015 | ≤0.015 | ≤0.015 |
| Burkholderia cepacia | 75 | 0.03 | 2 | 0.06 | 0.5 |
| species | | | | | |
| Providencia spp. | 74 | 0.06 | 32 | 0.5 | 2 |
| Achromobacter spp. | 40 | ≤0.015 | 0.5 | 0.06 | 0.12 |
| Legionella pneumophila | 35 | ≤0.015 | ≤0.015 | ≤0.015 | ≤0.015 |
| Campylobacter sp. | 33 | ≤0.015 | 0.03 | ≤0.015 | ≤0.015 |
| Shigella sp. | 30 | ≤0.015 | 0.25 | 0.03 | 0.06 |
| Helicobacter pylori | 25 | ≤0.015 | 0.5 | ≤0.015 | ≤0.015 |
| Enterobacter aerogenes | 3 | 0.25 | 0.25 | 0.25 | 0.25 |
| Hemophilus influenzae | 2 | ≤0.015 | ≤0.015 | ≤0.015 | ≤0.015 |
| Moraxella catarrhalis | 1 | ≤0.015 | ≤0.015 | ≤0.015 | ≤0.015 |
| Fusobacterium spp. | 33 | ≤0.015 | 0.5 | ≤0.015 | ≤0.015 |
| (anaerobe) | | | | | |
| Bacteroides fragilis | 31 | ≤0.015 | 8 | 0.06 | 0.25 |
| (anaerobe) | | | | | |
| Prevotella sp. (anaerobe) | 30 | ≤0.015 | 1 | ≤0.015 | ≤0.015 |
| Mycoplasma species | 35 | ≤0.015 | ≤0.015 | ≤0.015 | ≤0.015 |
| (atypical) | | | | | |
| Ureaplasma species | 10 | ≤0.015 | 0.03 | ≤0.015 | ≤0.015 |
| (atypical) | | | | | |
| GRAM-POSITIVE | | | | | |
| BACTERIA | | | | | |
| Staphylococcus aureus | 560 | <0.015 | 0.5 | 0.03 | 0.03 |
| Coagulase-negatives | 208 | <0.015 | 0.03 | <0.015 | <0.015 |
| Staphylococcus | | | | | |
| Enterococcus faecalis | 173 | <0.015 | 0.25 | 0.03 | 0.06 |
| Streptococcus pyogenes | 139 | 0.03 | 0.06 | 0.03 | 0.03 |
| Enterococcus faecium | 126 | <0.015 | 0.5 | 0.03 | 0.12 |
| Streptococcus | 122 | 0.03 | 0.06 | 0.03 | 0.03 |
| pneumoniae | | | | | |
| Clostridium difficile | 34 | <0.015 | 0.06 | <0.015 | 0.03 |
| (anaerobe) | | | | | |

TABLE 2
MIC₉₀ results against Bio-threat pathogens
for compound of Formula (I) formate salt

| | | | | MIC₅₀ | MIC₉₀ |
|---|---|---|---|---|---|
| Bio-Threat Pathogens | N | MIC Range (μg/ml) | | (μg/ml) | (μg/ml) |
| Bacillus anthracis | 42 | ≤0.015 | ≤0.015 | ≤0.015 | ≤0.015 |
| Burkholderia | 40 | ≤0.015 | 4 | 0.25 | 0.5 |
| pseudomallei | | | | | |
| Francisella tularensis | 36 | ≤0.015 | ≤0.015 | ≤0.015 | ≤0.015 |
| Burkholderia mallei | 36 | ≤0.015 | 4 | 0.25 | 1 |
| Yersinia pestis | 35 | ≤0.015 | ≤0.015 | ≤0.015 | ≤0.015 |

*N indicating number of bacterial strains tested in particular species.

### Example 3

### Salt Screening

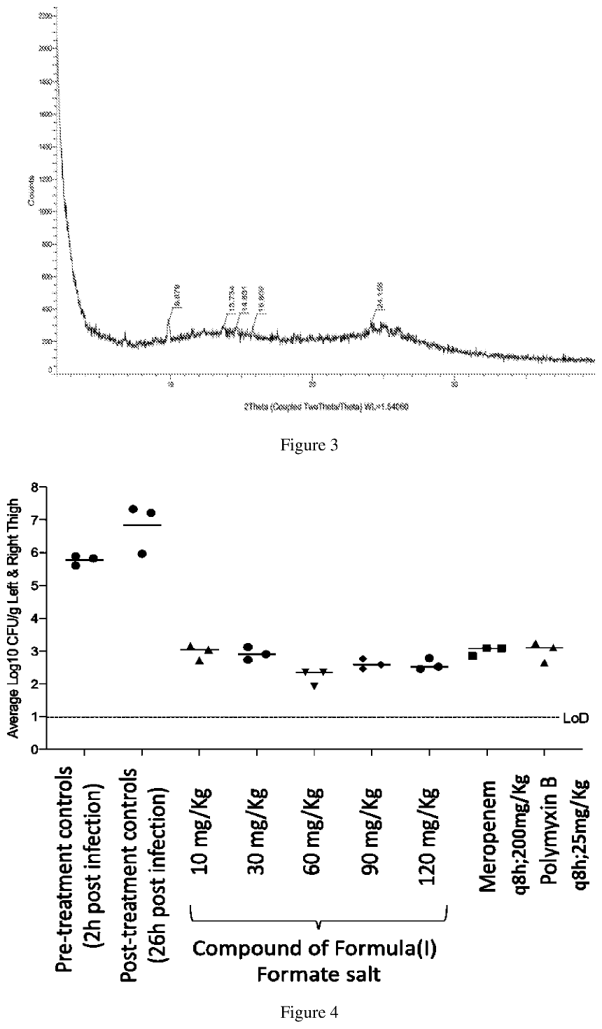

The salt screening of compound of Formula (I) was evaluated to select the suitable salt for the development of the formulations with improved solubility in water. The solid-state properties of the compound of Formula (I) were studied using pXRD diffraction pattern. The free amine of the compound of Formula (I) was found to be amorphous in nature by pXRD diffractogram (FIG. 3) as X-ray diffraction showed halo diffraction pattern at 2-40° (2 theta) range.

### Selection of Counterions

The free amine compound of Formula (I) was found to be soluble only in dichloromethane and methanol (1:1) mixture. Hence, the same solvent system was used during the salt screening experiments using slow solvent evaporation approach. The acidic counter ions were used to screen the salt forming propensity of compound of Formula (I) due to its weakly basic nature (pKa˜6.81). Theoretically, counter ions with pKa values at least less than 2 units than basic pKa value of the compound of Formula (I) were considered for the salt formation. Table 3 shows the list of selected counterions for salt screening.

TABLE 3

| Counter ion | pKa |
|---|---|
| p-toluene sulfonic acid | −1.34 |
| methane sulfonic acid | −2.6 |
| camphor sulfonic acid | 1.5 |
| maleic acid | 2.0, 6.26 |
| malonic acid | 2.8, 5.67 |
| malic acid | 3.4, 5.11 |
| succinic acid | 4.19, 5.57 |
| Fumaric acid | 3.03, 4.44 |
| oxalic acid anhydrous | 1.25, 4.23 |
| benzene sulfonic acid | −2.8 |
| ethane sulfonic acid | 1.68 |
| Ascorbic acid | 4.17 & 11.6 |
| Citric acid | 3.13, 4.76 & 6.40 |
| Lactic acid | 3.86 |
| Formic Acid | 3.75 |

A common solvent method in which drug and counter ion would be soluble was optimized and salt screening was performed. The qualitative solubility of counter ions was performed in water as solvent and the stoichiometrically of 1:1.125 ratio of drug and counter ion was used to prepare solutions during salt screening experiments.

Compound of Formula (I) (100 mg) was dissolved in suitable amount of organic solvent system containing dichloromethane and methanol (1:1) mixture and required amount of counter ion dissolved in methanol was added. Then, heated at 50° C. to 60° C. for few minutes and allowed to equilibrate to room temperature to observe if any salt formation occurred due to crystallization or precipitation from the solution. The results of salt screening is summarized in Table 4 below.

TABLE 4
Salt feasibility trials using free amine of compound of Formula(I)

| Acidic | |
|---|---|
| Counter ion | Observation |
| HCl | Solid was formed |
| p-toluene | Initially a solution was observed. Formed solid |
| sulfonic acid | after solvent evaporation. |
| methane | Initially a solution was observed. Formed solid |
| sulfonic acid | after solvent evaporation. |
| camphor | Solid formed |
| sulfonic acid | |
| maleic acid | PPt. formed after counterion addition. Solid formed. |
| malonic acid | PPt. formed after counterion addition. Solid formed. |
| Fumaric acid | PPt. formed after counterion addition. Solid formed. |
| tartaric acid | PPt. formed after counterion addition. Solid formed. |
| succinic acid | PPt. formed after counterion addition. Solid formed. |
| | But, gel formation observed |
| oxalic acid | PPt. formed after counterion addition. Solid formed. |
| anhydrous | |
| malic acid | PPt. formed after counterion addition. Solid formed. |
| citric acid | PPt. formed after counterion addition. Solid formed. |
| Ascorbic acid | Remained solid for 1-2 days, showed |
| | deliquescence later |
| Formic acid | Solid formed |

*Ppt—precipitate

The counter ions which yielded the crystalline salts from the experiments above were further considered for scale up trials (200 mg scale). Then, the selected crystalline salts were evaluated for solubility in various aqueous media to check solubility enhancement of the salts of the compounds of Formula (I).

Compound of Formula (I) as free amine (200 mg) was weighed into a round bottomed flask and dissolved in sufficient amount of dichloromethane and methanol (1:1) mixture with aid of sonication and slight heating on water bath at around 40-50° C. Then, the required amount of acidic counter ion was weighed (if solid) and dissolved in methanol or measured (if liquid counter ion) and then transferred to above contents in round bottomed flask and kept for stirring for overnight to observed solids precipitated and the solid was collected after complete solvent evaporation.

### Solubility of Selected Salts of Compound of Formula(I)

The as-prepared salts of the compound of Formula (I) were evaluated for qualitative solubility in various aqueous media to select the best counter ion based on solubility enhancement compared to formate salt. 2 mg equivalent salts of the compound of Formula (I) were weighed in a clear glass vial. To this, 20 μL solvent as listed in table 5 below was added and sonicated, vortexed and observed if dissolved. If not dissolved, further increments of the solvents were added as given in the Table 5. Sonication and vortexing were performed in an order to dissolve the compound at each increment.

TABLE 5
Qualitative solubility of various salts screened of compound of Formula (I)

| | | | | | | | | | | 10% | |
|---|---|---|---|---|---|---|---|---|---|---|---|
| Salt trial | | | | | | | 10% w/v | | 30% lactic | sodium | 10% |
| using | Normal saline | | Purified water | | PBS pH 7.2 | | ascorbic acid | | acid | saccharin | sucralose |
| acidic | | Qual. Sol | | Qual. Sol | | Qual. Sol | | Qual. Sol | Qual. Sol | Qual. Sol | Qual. Sol |
| counterion | pH | (mg/mL) | pH | (mg/mL) | pH | (mg/mL) | pH | (mg/mL) | (mg/mL) | (mg/mL) | (mg/mL) |
| Mesylate | 4.4 | <1 | 4.5 | <1 | 6.9 | <1 | 2.06 | <1 | <1 | <1 | <1 |
| trial | | | | | | | | | | | |
| Fumarate | 3.68 | <1 | 3.7 | <1 | 6.97 | <1 | 2.14 | May be | <1 | <1 | <1 |
| trial | | | | | | | | about 2.5 | | | |
| | | | | | | | | mg/mL, | | | |
| | | | | | | | | clear to | | | |
| | | | | | | | | translucent | | | |
| | | | | | | | | with | | | |
| | | | | | | | | particles | | | |
| HCl trial | 4.97 | <1 | 4.8 | <1 | 7.2 | <1 | 2.02 | About 1-5 | 5-10 | <1 | <1 |
| | | | | | | | | mg/mL, | mg/mL | | |
| | | | | | | | | clear with | | | |
| | | | | | | | | few | | | |
| | | | | | | | | particles | | | |
| Malate | 4.01 | <1 | 4.1 | <1 | 6.9 | <1 | 2.08 | About 1-5 | About 5-10 | <1 | <1 |
| trial | | | | | | | | mg/mL, | mg/mL | | |
| | | | | | | | | clear with | | | |
| | | | | | | | | few | | | |
| | | | | | | | | particles | | | |
| Ascorbate | 5.1 | <1 | 4.61 | <1 | 7.18 | <1 | 12.08 | <1 | 10 mg/mL | <1 | <1 |
| trial | | | | | | | | | translucent | | |
| | | | | | | | | | coloured | | |
| | | | | | | | | | solution | | |
| Formate | — | — | — | — | — | — | — | 10 mg/mL | 10 mg/mL | <1 | <1 |

Inference from Salt Screening:

To enhance the solubility of the compound of Formula (I), salt screening was performed to meet high solubility requirement for higher doses required for human dosing. Various salts were screened using acidic counter ions such as sulfonate, mesylate, succinate, tosylate, fumarate, formate, maleate, malate, oxalate, lactate, glucoronate and HCl. The preliminary feasibility trials yielded in amorphous salts in the case of maleate, citrate and oxalate trials. There was amorphous solid formation even in case of lactate and ascorbate salts. However, from Table 5 it could be identified that only the formate salt of the compound of Formula (I) exhibited crystallinity and increased solubility.

The formate salt of compound of Formula (I) resulted in 10 mg/mL solution in both 10% w/v ascorbic acid aqueous solution and 30% v/v lactic acid aqueous solution, whereas the HCl salt and malate salt resulted in clear solutions at 5 mg/mL concentration in 30% lactic acid aqueous solution.

### Saturation Solubility Determination of Formate Salt of Compound of Formula (I)

The saturation solubility of compound of Formula (I) formate salt was performed at 25° C.±2° C. in various vehicles, acids, additives as listed in Table 6A.

The water for injection (WFI) and various solvents (5 mL or 5.0 g) were taken in 10 mL clear glass vials. After adding an appropriate amount of compound of Formula (I) formate salt in each vial and was closed with stopper. These glass vials were attached in an orbital shaking water bath. The shaking was done for 72 hours, and the temperature maintenance was done at around 25±2° C. Then resulting test samples were evaluated for drug concentration determination in each concerned solvent at 24 hrs, 48 hrs and 72 hrs.

TABLE 6A
Saturation solubility results of compound
of Formula(I) formate salt

| Sr. | | Concentration (mg/mL) | | |
|---|---|---|---|---|
| No. | Solvent name | 24 Hrs | 48 Hrs | 72 Hrs |
| 1 | Water for Injection | 2.1103 | 2.6645 | 2.7013 |
| 2 | Propylene glycol | 1.1552 | 1.1028 | 1.1466 |
| 3 | Polyethylene glycol 400 (PEG 400) | 1.0979 | 0.9836 | 1.032 |
| 4 | Polysorbate 80 | 0.3855 | 0.2994 | 0.3551 |
| 5 | Polyethylene glycol 300 (PEG 300) | 1.1723 | 1.1057 | 1.0377 |
| 6 | Polysorbate 20 | 0.523 | 0.4543 | 0.5399 |
| 7 | Glycerin | 1.4971 | 1.1941 | 1.311 |
| 8 | Kolliphor ELP (Polyoxyl 35 | 0.2514 | 0.1691 | 0.2395 |
| | Castor oil) | | | |
| 9 | Ethanol | 0.2639 | 0.2702 | 0.2634 |
| 10 | 5% Ascorbic acid | 5.1256 | 11.6209 | 12.6575 |
| 11 | 1% Polyvinyl Pyrrolidone K-12 | 2.1051 | 2.4658 | 2.5368 |
| | (PVP K-12) | | | |
| 12 | 30% Hydroxypropyl β-Cyclodextrin | 2.5311 | 3.354 | 3.56 |
| | (HPβCD) | | | |
| 13 | 1.8% Lactic acid (90%) | 8.7099 | 8.8433 | 9.3364 |
| 14 | 4.4% Glacial Acetic acid | 7.3871 | 8.2251 | 8.754 |
| 15 | 5% Ascorbic acid + 30% HPβCD | 7.597 | 7.7072 | 11.8594 |
| 16 | 5% Ascorbic acid + 30% HPβCD + | 6.7077 | 7.3125 | 11.2995 |
| | 1% PVP K-12 | | | |
| 17 | 5% Ascorbic acid + 1.8% Lactic | 18.633 | 18.842 | 22.4482 |
| | acid + 4.4% Glacial Acetic acid + | | | |
| | 30% HPβCD + 1% PVP K-12 | | | |

The compound of Formula(I) formate salt exhibited very limited solubility in water and different water-soluble solvents however showed maximum solubility in individual acids like ascorbic acid, acetic acid, and lactic acid. The combination of acids along with hydroxy propyl β-cyclodextrin (HPP-CD) and polyvinylpyrrolidone (PVP) K-12 showed solubility enhancement for the compound of Formula (I) formate salt. It could be observed that a use of combination of ascorbic acid, lactic acid, glacial acetic acid with HPβCD and PVP K-12 resulted in maximum solubility of the compound of Formula (I) formate salt.

### pH Solution Stability Study

The pH solution stability was performed at 25° C.±2° C. in the below buffers listed in Table 6B (50 mM Acetate Buffer for pH 3.0, pH 4.0, pH 5.0 and 50 mM Phosphate buffer for pH 6.0).

For determining the pH solution stability of the compound of Formula (I) formate salt at different pH range, the respective buffer was prepared by dissolving the buffer salts in WFI. The weighed quantity of compound of Formula (I) formate salt was added to each buffers system and dissolved. Volume make up was performed with WFI. The 5 mL of sample was filled in 5 mL USP Type I Vials. Vials were stopped and sealed. These vials were kept at 25° C. for stability study. The blank buffers/placebo samples were also placed along with active samples. The samples were evaluated for description, pH, assay, related substances at 24 Hrs, 48 Hrs and 72 Hrs time points.

TABLE 6B
Compositions for pH Solution Stability Study of Compound
of Formula (I) formate salt (Batch Size: 50 mL each)

| Sr. | | Samples/Quantity (mg/mL) | | | |
|---|---|---|---|---|---|
| No. | Ingredients | SFA | SFB | SFC | SFD |
| 1 | Compound of formula | 0.5 | 0.5 | 0.5 | 0.5 |
| | (I) formate salt | | | | |
| 2 | Glacial Acetic Acid | 2.95 | 2.549 | 1.081 | NA |
| 3 | Sodium acetate | 0.119 | 1.027 | 4.355 | NA |
| | trihydrate | | | | |
| 4 | Monosodium | NA | NA | NA | 5.947 |
| | Phosphate | | | | |
| | Monohydrate | | | | |
| 5 | Disodium Phosphate | NA | NA | NA | 0.98 |
| | Anhydrous | | | | |
| 6 | Water For Injection | 1 mL | 1 mL | 1 mL | 1 mL |
| | (Qs to) | | | | |
| 7 | Target pH | pH 3.0 | pH 4.0 | pH 5.0 | pH 6.0 |

NA: Not Applicable

TABLE 6C
Results of pH Solution Stability Study of formulations in Table 6B

| | | SFA sample | | | | SFB sample | | | |
|---|---|---|---|---|---|---|---|---|---|
| | | (50 mM Acetate Buffer, pH 3.0) | | | | (50 mM Acetate Buffer, pH 4.0) | | | |
| | | | 24 | 48 | 72 | | 24 | 48 | 72 |
| Sr. No. | Test | I | Hrs | Hrs | Hrs | I | Hrs | Hrs | Hrs |
| 1 | Description | * | * | * | * | * | * | * | * |
| 2 | pH | 3.28 | 3.37 | 3.32 | 3.34 | 3.9 | 3.98 | 3.95 | 3.93 |
| 3 | Assay (%) | 102.5 | 99.4 | 98.7 | 99.1 | 100.4 | 98.1 | 98.4 | 97.8 |
| 4 | Related | | | | | | | | |
| | Substances (%) | | | | | | | | |
| | Highest | 0.39 | 0.33 | 0.98 | 1.6 | 0.4 | 0.37 | 1.31 | 1.65 |
| | Unknown | | | | | | | | |
| | Total Unknown | 1.45 | 1.39 | 2.51 | 3.1 | 1.54 | 1.39 | 2.69 | 2.97 |
| | Total impurities | 1.5 | 1.4 | 2.5 | 3.1 | 1.5 | 1.4 | 2.7 | 3 |

I: Initial,
*Clear Pale Yellowish Solution

TABLE 6D
Results of pH Solution Stability Study of formulations in Table 6B

| | | SFC | | | | SFD | | | |
|---|---|---|---|---|---|---|---|---|---|
| | | (50 mM Acetate Buffer, pH 5.0) | | | | (50 mM Phosphate Buffer, pH 6.0) | | | |
| Sr. | | | 24 | 48 | 72 | | 24 | 48 | 72 |
| No. | Test | I | Hrs | Hrs | Hrs | I | Hrs | Hrs | Hrs |
| 1 | Description | * | * | * | * | ** | ** | ** | ** |
| 2 | pH | 4.84 | 4.95 | 4.92 | 4.89 | 6 | 6.1 | 6.07 | 6.07 |
| 3 | Assay (%) | 99.0 | 97.3 | 97.6 | 97.6 | 4.9 | 4.5 | 4.4 | 4.6 |
| 4 | Related Substances | | | | | | | | |
| | (%) | | | | | | | | |
| | Highest Unknown | 0.28 | 0.29 | 1.09 | 1.47 | 3.8 | 4.13 | 4.14 | 4.41 |
| | Total Unknown | 1.24 | 1.22 | 2.21 | 2.73 | 18.13 | 17.73 | 18.2 | 18.17 |
| | Total impurities | 1.2 | 1.2 | 2.2 | 2.7 | 18.1 | 17.7 | 18.2 | 18.2 |

I: Initial,
*Clear Pale Yellowish Solution;
**Off-white suspension

At pH 3.0, 4.0 and 5.0 the formulation was found to be a clear pale yellow color solution, however at pH 6.0 it was a off-white suspension. There was drop in assay at all pH range after 24 Hrs at 25° C. The drop was between 0.4% to 3.5%. This indicated that compound of Formula (I) formate has solution stability upto 24 Hrs at pH 3.0-pH 5.0. Related substances (RS) data indicated increase in impurity level after 48 Hrs at 25° C. The increase in RS was significant at pH 6.0 compared to pH 3.0, 4.0 and 5.0. Hence for the formulation development the target pH range would be between pH 3.0 to 5.0.

### Example 4

### Compatibility Study

The drug herein is referred to as is the active component which is the compound of Formula (I) or its salt thereof. The compatibility study of the drug with the formulation components was carried out to identify the suitable formulation component in achieving the acceptable impurity profile. To achieve this, compatibility and stability studies were conducted in various components in solution/suspension state like buffers, solvents, surfactant mixtures at 25° C./60% RH, and 40° C./75% RH for 1 month. All these batches were charged for the stability at mixtures at 25° C./60% RH, and 40° C./75% RH for 1 month.

The following Table 7 provides details of container closure systems used in the present disclosure.

TABLE 7
Details of container closure systems

| Sr. No. | Primary Packing material |
|---|---|
| 1 | Vial: 10 mL clear Tubular glass vial |
| 2 | Rubber stopper: 20 mm Gray Omniflex plus |
| | (fluoro coated) stopper V9048 FM 259/0 |
| 3 | Seal: 20 mm Aluminium Tear-off seal |

### Formulation for Compatibility Study

Various formulations F1 to F5 were prepared using the components as described in Table 8 below. Further the stability and solubility studies were carried out for each formulation and the results are tabulated in Tables 9, 10 and 11.

TABLE 8
Formulation for drug compatibility study

| | | Quantity/batch (Batch Size = 40 mL) | | | | | | | | | |
|---|---|---|---|---|---|---|---|---|---|---|---|
| Sr. No. | Ingredients | F1 | | F2 | | F3 | | F4 | | F5 | |
| 1 | Compound of | 1.20 | g | 1.20 | g | 1.20 | g | 1.20 | g | 1.20 | g |
| | formula (I) | | | | | | | | | | |
| | formate salt | | | | | | | | | | |
| | (Concentration: | | | | | | | | | | |
| | 30 mg/mL) | | | | | | | | | | |
| 2 | Ascorbic acid | 2.00 | g | X | | X | | X | | 2.00 | g |
| 3 | Lactic acid | X | | 0.720 | g | X | | X | | 0.720 | g |
| 4 | Glacial Acetic | X | | X | | 1.76 | g | X | | 1.76 | g |
| | acid | | | | | | | | | | |
| 5 | L-Arginine | X | | X | | X | | 2.50 | g | 2.50 | g |
| 6 | WFI (Qs to) | 40 | mL | 40 | mL | 40 | mL | 40 | mL | 40 | mL |

*X indicates absence of that particular component

TABLE 9
Results of drug-components compatibility study of F1 and F2

| | | Stability Conditions | | | | | | | | | |
|---|---|---|---|---|---|---|---|---|---|---|---|
| | | F1 | | | | | F2 | | | | |
| | | (Ascorbic acid) | | | | | (Lactic acid) | | | | |
| | | | 25° C. ± 2° C./ | | 40° C. ± 2° C./ | | | 25° C. ± 2° C./ | | 40° C. ± 2° C./ | |
| | | | 60 ± 5% RH | | 75 ± 5% RH | | | 60 ± 5% RH | | 75 ± 5% RH | |
| Sr. | Test | | 15 | 30 | 15 | 30 | | 15 | 30 | 15 | 30 |
| No. | Performed | Initial | Days | Days | Days | Days | Initial | Days | Days | Days | Days |
| 1 | Description | * | * | * | # | # | * | * | * | ## | ## |
| 2 | Assay | 97.8 | 98.6 | 94.3 | 95.1 | 92.6 | 99.3 | 98.6 | 97.4 | 93.7 | 97.2 |
| | (%) | | | | | | | | | | |
| 3 | Related | | | | | | | | | | |
| | Substances | | | | | | | | | | |
| | (%) | | | | | | | | | | |
| | Highest | 0.127 | 0.23 | 0.39 | 0.49 | 1.04 | 0.12 | 0.20 | 0.3 | 0.33 | 0.78 |
| | Unknown | | | | | | | | | | |
| | Total | 0.455 | 0.78 | 1.66 | 1.38 | 2.67 | 0.369 | 0.88 | 1.62 | 1.22 | 2.50 |
| | Unknown | | | | | | | | | | |
| | Total | 0.455 | 0.80 | 1.70 | 1.40 | 2.70 | 0.369 | 0.90 | 1.60 | 1.20 | 2.50 |
| | impurities | | | | | | | | | | |

*Pale Yellow Suspension;
#Light brick red color solution with brownish particles
##Yellow color suspension with green pellets

TABLE 10
Results of Drug-Excipients compatibility study of F3 and F4

| | | Stability Conditions | | | | | | | | | |
|---|---|---|---|---|---|---|---|---|---|---|---|
| | | F3 | | | | | F4 | | | | |
| | | (Glacial Acetic acid) | | | | | (L-Arginine) | | | | |
| | | | 25° C. ± 2° C./ | | 40° C. ± 2° C./ | | | 25° C. ± 2° C./ | | 40° C. ± 2° C./ | |
| | | | 60 ± 5% RH | | 75 ± 5% RH | | | 60 ± 5% RH | | 75 ± 5% RH | |
| Sr. | Test | | 15 | 30 | 15 | 30 | | 15 | 30 | 15 | 30 |
| No. | Performed | Initial | Days | Days | Days | Days | Initial | Days | Days | Days | Days |
| 1 | Description | * | ## | * | ## | ## | * | ## | ## | ## | ## |
| 2 | Assay | 97.9 | 98.5 | 83.0 | 92.3 | 96.3 | 95.2 | 82.4 | 55.7 | 82.4 | 84.7 |
| | (%) | | | | | | | | | | |
| 3 | Related | | | | | | | | | | |
| | Substances | | | | | | | | | | |
| | (%) | | | | | | | | | | |
| | Highest | 0.095 | 0.18 | 0.3 | 0.31 | 0.74 | 0.756 | 2.12 | 3.04 | 5.44 | 6.79 |
| | Unknown | | | | | | | | | | |
| | Total | 0.337 | 0.87 | 1.63 | 1.14 | 2.79 | 1.274 | 3.19 | 5.86 | 8.07 | 12.53 |
| | Unknown | | | | | | | | | | |
| | Total | 0.337 | 0.90 | 1.60 | 1.10 | 2.80 | 1.274 | 3.20 | 5.90 | 8.10 | 12.5 |
| | impurities | | | | | | | | | | |

*Pale Yellow Suspension;
##Yellow color suspension with green pellets

TABLE 1
Results of drug-components compatibility study of F5

| | | Stability Condition | | | | |
|---|---|---|---|---|---|---|
| | | F5 | | | | |
| | | (Formulation with all components) | | | | |
| | | | 25° C. ± 2° C./ | | 40° C. ± 2° C./ | |
| | | | 60 ± 5% RH | | 75 ± 5% RH | |
| Sr. No. | Test Performed | Initial | 15 Days | 30 Days | 15 Days | 30 Days |
| 1 | Description | * | @@@@ | @@@@ | @@@@ | @@@@ |
| 2 | Assay | 100.8 | 95.2 | 95.9 | 94.4 | 92.6 |
| 3 | Related Substances | | | | | |
| | Highest Unknown | 0.233 | 0.22 | 0.3 | 0.64 | 1.03 |
| | Total Unknown | 0.779 | 1.05 | 1.61 | 1.54 | 2.94 |
| | Total impurities | 0.779 | 1.1 | 1.6 | 1.5 | 2.9 |

*Pale Yellow Suspension;
@@@@Thick brick red colour suspension

In the drug-components compatibility study, significant changes in appearance, assay and relative substance (RS) were observed at both conditions i.e. at 25° C.±2° C./60±5% RH and 40° C.±2° C./75±5% RH as depicted in Table 9, Table 10 and Table 11. The change in appearance (colour of solution/suspension) was observed mainly due to ascorbic acid discoloration under both conditions, as placebo (formulation without compound of Formula (I)) samples containing ascorbic acid have also shown the discoloration. The drop in the assay was more in the case of batches placed as formulations compared to the assay of batches placed as with individual components except for the batch with L-arginine (F4).

The impurity profile in batches with individual components (F1, F2, F3 and F4) were found to be lesser compared to the batches manufactured as formulations (F5).

### Solubility Enhancement of Compound of Formula (I) or its Salt

The compound of Formula (I) formate salt had limited solubility in water and further to be improved to achieve the target formulation strength. This was achieved by using combinations of acids like ascorbic acid, lactic acid and glacial acetic acid.

The compound of Formula (I) formate salt was unstable at higher pH range i.e., at neutral to alkaline pH range (pH 5.0 to 10.0) and very stable at acid pH (<5). Hence, the desired target pH range for the formulations comprising the compound of Formula (I) formate salt is between pH 3.0 to pH 5.0. The compound of Formula (I) formate salt was highly unstable in a liquid state (Solution/Suspension); hence the drug formulations were manufactured as lyophilized drug formulations to achieve a stable product.

The different prototype formulations were considered to improve the solubility Compound of Formula (I) formate salt using combination of acids. The vortexing and mixing at 35° C.-40° C. was performed to solubilize the compound of Formula (I) formate salt. All observations were recorded once the solution temperature reached to 25° C.-30° C.

Among the solubilizers studied, the compound of Formula(I) formate salt was most soluble in acidic conditions and in combination of acids like ascorbic acid, lactic acid and acetic acid. Also, with the addition of solubilizers like hydroxypropyl 0-CD and polyvinyl pyrrolidine K-12, the solubility was enhanced to greater than 25 mg/mL.

TABLE 12
Results of solubility enhancement of compound of Formula(I) formate salt.

| | | | Approx. | | |
|---|---|---|---|---|---|
| | | | Quantity of | | |
| | | | compound of | | |
| | | | Formula(I) | | |
| | | | formate salt | | |
| Sr. | | | solubilized | | |
| No. | Batch No. | Trial Details/composition | (mg/mL) | pH | Remark |
| 1 | SF19000361 | 5% Ascorbic acid solution | 5 | −NR | Clear solution |
| 2 | | 7.5% Citric acid solution | 5-10 | 1.96 | PPt on standing |
| 3 | | 4.5% Acetic acid Solution | 5-10 | −NR | Clear solution |
| 4 | | 1.5% Succinic acid | 5 | NR | Clear solution |
| | | Solution | | | |
| 5 | | 7.5% Citric acid + 20% | 5-10 | NR | PPt on standing immediately |
| | | Sulfobutyl ether β-CD | | | |
| | | Solution | | | |
| 6 | | 7.5% Citric acid + 20% | 15 | 1.94 | PPt on standing for |
| | | HPβ-CD Solution | | | overnight |
| 7 | | 7.5% Citric acid + 20% | 15-20 | NR | PPt on standing for |
| | | HPβ-CD + 4% Ethanol | | | overnight |
| | | Solution | | | |
| 8 | | 5% Ascorbic acid + 5% | 10-15 | 2.14 | PPt on standing immediately |
| | | Citric acid + 0.5% PVP K- | | | |
| | | 12 + 20% HPβ-CD Solution | | | |
| 9 | | 10% Ascorbic acid + 1% | 25-30 | 2.9 | 30 mg/mL sample- slightly |
| | | PVP K-12 + 20% HPβ-CD | | | hazy Solution |
| | | Solution | | | 25 mg/mL sample- Clear |
| | | | | | pale yellow Solution |
| | | | | | On standing for overnight |
| | | | | | similar observation |
| 10 | | 5% Ascorbic acid + 2% | 15-20 | 3.19 | Clear solution |
| | | PVP K-12 + 20% HPβ-CD | | | |
| | | Solution | | | |
| 11 | | 7.5% Ascorbic acid + 2% | 20-25 | 3.08 | No Significant effect of |
| | | PVP K-12 + 20% HPβ-CD | | | PVP K-12 concentration |
| | | Solution | | | on solubility of compound |
| | | | | | formula(I)formate salt |
| 12 | | 7.5% Ascorbic acid + 1% | 20-25 | 3.07 | Clear solution |
| | | PVP K-12 + 20% HPβ-CD | | | |
| | | Solution | | | |
| 13 | | 5% Ascorbic acid + 1.5% | 15-20 | 2.88 | — Clear solution |
| | | Lactic acid + 1% PVP K- | | | |
| | | 12 + 20% HPβ-CD Solution | | | |
| 14 | SF19000376 | 5% Ascorbic acid + 1% | 15-20 | 3.25 | Significant effect of |
| | | PVP K-12 + 30% HPβ-CD | | | Ascorbic acid concentration |
| | | Solution | | | on solubility of compound |
| | | | | | formula(I)formate salt |
| 15 | | 7.5% Ascorbic acid + 1% | 20-25 | 3.15 | Clear solution |
| | | PVP K-12 + 30% HPβ-CD | | | |
| | | Solution | | | |
| 17 | | 10% Ascorbic acid + 1% | 30-35 | 3.03 | Clear solution |
| | | PVP K-12 + 30% HPβ-CD | | | |
| | | Solution | | | |
| 18 | | 7% Ascorbic acid + 4.4% | 35 | 3.06 | Clear Solution with few |
| | | Acetic acid + 1% PVP K- | | | undissolved dust like |
| | | 12 + 30% HPβ-CD Solution | | | particles |
| 19 | | 7% Ascorbic acid + 0.9% | 15-20 | 3.25 | Clear solution |
| | | Glycine + 30% HPβ-CD | | | |
| | | Solution | | | |
| 20 | | 7% Ascorbic acid + 4.4% | 30 | 3.16 | Clear Solution with few |
| | | Acetic acid + 0.9% | | | undissolved dust like |
| | | Glycine + 30% HPβ-CD | | | particles |
| | | Solution | | | |
| 21 | | 7% Ascorbic acid + 1.8% | 25-30 | 2.91 | Clear solution |
| | | lactic acid + 1% PVP K- | | | |
| | | 12 + 30% HPβ-CD Solution | | | |
| 22 | SF19000388 | 6% Ascorbic acid + 4.4% | 35 | 2.84 | Clear Pale Yellow color |
| | | Acetic acid + 1.8% lactic | | | solution. |
| | | acid + 1% PVP K-12 + | | | On standing for overnight |
| | | 30% HPβ-CD Solution | | | similar observation |
| 23 | | Acetate Buffer (200 mM, | 35 | 2.78 | Slightly Hazy Solution |
| | | pH 3.0) + 6% Ascorbic | | | |
| | | acid + 1.8% lactic acid + | | | |
| | | 1% PVP K-12 + 30% HPβ- | | | |
| | | CD Solution | | | |
| 24 | SF19000413 | 5% Ascorbic acid + 4.4% | 30 | 3.73 | Clear Pale Yellow Solution |
| | | Acetic acid + 1.8% lactic | | | pH is adjusted by using L- |
| | | acid + 1% PVP K-12 + | | | Arginine Solution |
| | | 30% HPβ-CD Solution | | | |

NR: Not Recorded

### Use of L-Arginine as pH1 Adjusting Agent and Evaluation of Formulations

L-Arginine was used as a pH adjusting agent and the formulations were evaluated to maintain the formulation pH between 3.0 to 4.0. Two different batches of compound of Formula (I) formate salt (SF19000421 & SF19000435) of 10 mL each were prepared. The formulations prepared for evaluations are shown in Table 13. Formulation 35A comprised 1-arginine in addition to components of Formulation 21A.

TABLE 13
Formulation (21A) for pH solution stability
study of compound of Formula (I) formate salt

| Sr. | | Weight % | |
|---|---|---|---|
| No. | Ingredients | (% w/v) | Quantity(mg/mL) |
| 1 | Compound of formula (I) | 3 | 30 |
| | formate salt | | |
| 2 | Ascorbic Acid | 5 | 50 |
| | (solubilising agent) | | |
| 3 | Glacial Acetic Acid | 4.4 | 44 |
| | (solubilising agent) | | |
| 4 | Lactic acid 90% | 1.8 | 18 |
| | (solubilising agent) | | |
| 5 | Kollidon K12 PF (PVP K12) | 1.0 | 10 |
| 6 | HPβCD | 3.0 | 30 |
| 7 | L-Arginine (pH modifier) | qs | Qs |
| 8 | Milli-Q Water | qs | Qs to 1 mL |

TABLE 14
Observations of pH solution stability
study of compound of formula (I)

| Sr. | | | Observations | |
|---|---|---|---|---|
| No. | B. No. | Test | Initial | After 18-20 Hrs |
| 1 | 21A (As such) | Description | * | * |
| | | pH | 2.79 | 2.8 |
| | | pH | 3.29 | 3.24 |
| 6 | 35A (L-Arginine) | Description | * | * |
| | | pH | 4.07 | 4.05 |
| | | pH | 3.96 | 3.97 |

*Clear Pale Yellow Colour Solution

Table 14 illustrate the solution stability initially and after 18-20 hours of preparation of formulation. It could be seen that L-arginine was showing stable pH and the appearance of solution remained clear up to 18 hrs. Hence L-Arginine was found to be suitable for use as pH adjusting agent for the formulations of compound of Formula (I).

Evaluation of pH Modifier in Combination with Hydrotropic Agent

Formulations with pH modifier (pH adjusting agents) and hydrotropic agent (presence and absence) were evaluated to have pH of formulation between 3.5 to 4.0. Table 15 provides the formulations for solution stability at varying pH range of compound of Formula(I) formate salt and their corresponding stability studies are shown in Table 16.

TABLE 15
Formulations for solution stability at varying
pH range of compound of Formula(I) formate salt

| Sr. | | Samples/Quantity in mg/mL(wt %) | | |
|---|---|---|---|---|
| No. | Ingredients | 465A | 465B | 465C |
| 1 | Compound of Formula | 30 (3%) | 30(3%) | 30(3%) |
| | (I) formate salt | | | |
| 2 | Ascorbic Acid | 50(5%) | 50(5%) | 50(5%) |
| 3 | Glacial Acetic Acid | 44(4.4%) | 44(4.4%) | 44(4.4%) |
| 4 | Lactic acid 90% | 18(1.8%) | 18(1.8%) | 18(1.8%) |
| 5 | Kollidon K12 PF | 10(1%) | 10(1%) | 10(%) |
| | (PVP K12) | | | |
| 6 | Niacinamide | — | 25(2.5%) | 25(2.5%) |
| 7 | L-Arginine | 40(4%) | 31(3.1%) | 68.25(6.825%) |
| 8 | NaOH (1N) | — | — | — |
| 9 | Water for Injection | 1 mL | 1 mL | 1 mL |
| | (Qs to) | | | |

% expressed in w/v

TABLE 16
Observations of solution stability at different
pH range of compound of Formula(I) formate salt

| Sr. | | Observations | | |
|---|---|---|---|---|
| No. | Test | 465A | 465B | 465C |
| 1 | Appearance (Initial) | * | * | * |
| 2 | pH (Initial) | 3.52 | 3.54 | 3.95 |
| 3 | Appearance on standing | | | |
| | At RT | 2 Hrs | 20 Hrs | 2 Hrs |
| | | ** | * | ** |
| | At 30°-35° C. | 20 Hrs | 20 Hrs | 20 Hrs |
| | | *** | *** | ** |

*Clear Pale Yellow colour Solution;
**Pale Yellow ppt;
***Dark Yellow to brownish yellow ppt.
#Pale Yellow ppt immediately.

Of the pH adjusting agents and solubilizers evaluated, L-arginine and niacinamide combination showed the better stability (appearance of solution) up to 3 to 4 hrs and up to 20 hours (formulation 465B). Hence 1-arginine and niacinamide was used as pH adjusting agent, and hydrotropic agent, respectively. It could be further observed that the pH adjusting agent should be taken in a specific weight % range i.e., in a range of 0.5 to 30% and when taken outside said range resulted in precipitation of the formulation.

### Evaluation of Concentration of Acid (Solubilising Agent) to Solubilize the Compound of Formula (I) Formate Salt.

The formulation prepared using different acid concentrations were evaluated to obtain solubilization of the desired concentration of the compound of formula (I) formate salt as shown in Table 17 and their observations are tabulated in Table 18 below.

TABLE 3
Formulations of varying acid concentration to solubilize the compound of Formula (I) formate salt.

| Sr. | | Range | Quantity (mg/mL) | | | | | | | | |
|---|---|---|---|---|---|---|---|---|---|---|---|
| No. | Ingredients | (mg) | 470A | 470B | 470C | 470D | 470E | 470F | 470G | 470H | 470I |
| 1 | Compound of formula | 25 | 25 | 25 | 25 | 25 | 25 | 25 | 25 | 25 | 25 |
| | (I) formate salt | (2.5%) | | | | | | | | | |
| 2 | Ascorbic Acid | 41.67 | 41.67 | 41.67 | 41.67 | 41.67 | 41.67 | 41.67 | 41.67 | 41.67 | 41.67 |
| | | (4.167%) | | | | | | | | | |
| 3 | Glacial Acetic Acid | 12.22- | 36.67 | 24.44 | 12.22 | 36.67 | 24.44 | 12.22 | 36.67 | 24.44 | 12.22 |
| | | 36.67(1.22- | | | | | | | | | |
| | | 3.667% | | | | | | | | | |
| 4 | Lactic acid 90% | 10-15(1- | 15 | 15 | 15 | 10 | 10 | 10 | 12.5 | 12.5 | 12.5 |
| | | 1.5% | | | | | | | | | |
| 5 | Kollidon K12 PF (PVP | 10(1%) | 10 | 10 | 10 | 10 | 10 | 10 | 10 | 10 | 10 |
| | K12) | | | | | | | | | | |
| 6 | Niacinamide | 20.83(2.83%) | 20.83 | 20.83 | 20.83 | 20.83 | 20.83 | 20.83 | 20.83 | 20.83 | 20.83 |
| 7 | L-Arginine | 40- | 58.33 | 55 | — | — | — | — | 44 | 40 | — |
| | | 58.33(4- | | | | | | | | | |
| | | 5.833%) | | | | | | | | | |
| 8 | Water for Injection (Qs | 1 mL | 1 mL | 1 mL | 1 mL | 1 mL | 1 mL | 1 mL | 1 mL | 1 mL | 1 mL |
| | to) | | | | | | | | | | |

TABLE 18
Observations of optimization of acid concentration to
solubilize the compound of Formula (I) formate salt.

| Sr. | | Observations | | | | | | | | |
|---|---|---|---|---|---|---|---|---|---|---|
| No. | Test | 470A | 470B | 470C | 470D | 470E | 470F | 470G | 470H | 470I |
| 1 | Appearance | * | * | ** | ** | *** | *** | * | ** | *** |
| | (Initial) | | | | | | | | | |
| 2 | pH (Initial) | 3.91 | 3.93 | 2.53 | 2.54 | 2.6 | 2.63 | 3.94 | 3.97 | 2.67 |
| 3 | Appearance on | 20 Hrs | 20 Hrs | — | — | — | — | 16 Hrs | 16 Hrs | — |
| | standing | # | # | | | | | ## | ## | |

* Clear Pale Yellow color Solution;
** Hazy Pale Yellow colour solution;
*** Turbid Yellow colour solution;
# Pale Yellow ppt;
## Clear solution with few particles.

The concentration of glacial acetic acid and lactic acid had significant impact on the solubilization of compound of Formula (I). The formulations 470B and 470G had shown solubility of compound of Formula (I) formate salt at a concentration of 25 mg/mL. In the remaining formulations of Table 17, the compound of Formula (I) formate salt was not able to solubilize completely. The formulation 470A was considered as the control sample. Hence, minimum concentration of acids as shown in Table 19, was required to solubilize the compound of Formula (I) formate salt at different concentrations.

TABLE 19
Composition for minimum concentration of acid required
to solubilize the compound of formula (I) formate salt.

| | | Compound of | Compound of |
|---|---|---|---|
| | | formula (I) | formula (I) |
| Sr. | | 25 | 30 |
| No. | Ingredients | mg/mL(2.5%) | mg/mL(3.0%) |
| 1 | Ascorbic acid (mg/mL) | 41.67(4.167%) | 50(5%) |
| 2 | Glacial acetic acid (mg/mL) | 24.44(2.444%) | 29.5(2.95% |
| 3 | Lactic Acid (mg/mL) | 15(1.5%) | 18(1.8%) |

Based on improved solubility observed with organic acids, the formulations were further evaluated by use of the combinations of ascorbic acid and lactic acid to increase the solubility, reduce the overall acid strength with desired pH range between 4.00 to 4.50.

Evaluation of Solubility of Compound of Formula (I) Formate Salt (without Acetic Acid)

The formulations comprising the compound of Formula (I) formate salt was prepared with varying concentrations of the lactic acid as solubilizer and without acetic acid as shown in Table 20. The as-prepared formulations were observed for their appearance, pH and recorded in Table 21.

TABLE 4
Formulations for solubility study of compound
of Formula (I) formate salt without acetic acid

| Sr. | | Quantity (mg/mL) | | |
|---|---|---|---|---|
| No. | Ingredients | 528A | 528B | 528C |
| 1 | Compound of Formula | 25(2.5%) | 25(2.5%) | 20(2%) |
| | (I) formate salt | | | |
| 2 | Ascorbic Acid | 40(4%) | 40(4%) | 40(4%) |
| 3 | Lactic acid 90% | 18(1.8%) | 20(2%) | 18(1.8%) |
| 4 | Niacinamide | — | 25(2.5%) | 25(2.5%) |
| 5 | L-Arginine | — | 50(5%) | 60(6%) |
| 6 | Water For Injection | 1 mL | 1 mL | 1 mL |
| | (Qs to) | | | |

TABLE 21
Observations of solubility study of compound of
Formula (I) formate salt without acetic acid

| Sr. | | Observations | | | | | |
|---|---|---|---|---|---|---|---|
| No. | Ingredients | 528A | | 528B | | 528C | |
| 1 | Appearance (Before | Turbid | | Clear Pale | | Clear Pale | |
| | pH adjustment) | Solution | | Yellow colour | | Yellow colour | |
| | | | | Solution | | Solution | |
| 2 | Temperature for API | 35°-40° | C. | 35°-40° | C. | 35°-40° | C. |
| | Solubilization | | | | | | |
| 3 | Time Required for API | 180 | min | 120 | min | 20 | min |

| | | | | |
|---|---|---|---|---|
| 4 | Final pH of Solution | — | 4.49 | 5.3 |
| 5 | Appearance (After | — | Clear Pale | Turbid Yellow |
| | pH adjustment) | | Yellow colour | color solution |
| | | | Solution | |

From Table 21 above it could be seen that the compound of Formula (I) solubilized at a concentration of 20 mg/mL using combination of ascorbic acid and lactic acid and the pH of solution was found to be in the range pH 4.5 to 5.3. Therefore, it was inferred that acetic acid may be used as a solubilizer optionally.

### Example 5

### Evaluation of Lyophilization Cycle for Compound of Formula (I) Formate Salt

The lyophilization cycle for the formulations of the present disclosure were evaluated based on the lyophilization cycle of the formulations with two acid combinations i.e. ascorbic acid and lactic acid. The formulations were prepared using batch size of 100 ml each and the formulations are shown in Table 22.

TABLE 22
Formulation for lyophilization cycle development
of compound of Formula (I)

| | |
|---|---|
| Sr. | Samples/Quantity (mg/mL) |

| No. | Ingredients | 529A | 529B |
|---|---|---|---|
| 1 | Compound of formula | 20 (2%) | 20(2%) |
| | (I) formate salt | | |
| 2 | Ascorbic Acid | 40(4%) | 40(4%) |
| 3 | Lactic acid 90% | 18(1.8%) | 18(1.8%) |
| 4 | Mannitol | — | 20(2%) |
| 5 | Niacinamide | 25(2.5%) | 25(2.5%) |
| 6 | L-Arginine | 50(5%) | 50(5%) |
| 7 | Milli-Q Water (Qs to) | 1 mL | 1 mL |
| 8 | Target pH | pH 4.50 | pH 4.50 |

The formulations of Table 22 were prepared as described herein. Ascorbic acid (solubilising agent) was dissolved in water for Injection (˜70% of batch size, 25° C.-30° C.) followed by addition of lactic acid (solubilising agent) and was dissolved by stirring for 5 mins to obtain a solution. The solution was heated to 35° C.-40° C. Compound of Formula (I) formate salt was added in the solution and dissolved by stirring for 2 to 3 hrs. This solution was then cooled to room temperature on standing under continuous stirring. Niacinamide (hydrotropic agent) was added to the first solution and dissolved by stirring for 10 min to obtain a second solution. L-Arginine was added to the above solution and dissolved by stirring for 10 min to obtain the formulation. Mannitol (as per respective batch) was then added and dissolved under stirring for 10 min. The volume of solution was made up to batch size using Water for Injection (25°-30° C.) (vehicle) and mixed for 15 min.

The solution was filtered through 0.2μ PES filter (47 mm) using vacuum filtration assembly. The 25.0 mL of filtered solution was filled in 50 mL Clear USP type I Tubular vials, half stopper the vial and loaded for lyophilization. The lyophilization cycle was carried out as illustrated in Table 23 below.

TABLE 5
Lyophilization cycle for Batch No 529 of Table 22

| | | | | |
|---|---|---|---|---|
| Steps | Temp (° C.) | Time (min) | Vacuum (mT) | R/H |

Freezing

| | | | | |
|---|---|---|---|---|
| Step 1 | 0 | 30 | — | R |
| Step 2 | 0 | 60 | — | H |
| Step 3 | −45 | 50 | — | R |
| Step 4 | −45 | 720 | — | H |

Extra Freezing

| | |
|---|---|
| Freeze | −45 |
| Extra Freeze | 30 |
| Condenser | −40 |
| Vacuum | 500 |

Primary Drying

| | | | | |
|---|---|---|---|---|
| Step 1 | −30 | 60 | 200 | R |
| Step 2 | −30 | 1800 | 200 | H |
| Step 3 | −25 | 60 | 200 | R |
| Step 4 | −25 | 1800 | 200 | H |
| Step 5 | −20 | 60 | 100 | R |
| Step 6 | −20 | 900 | 100 | H |
| Step 7 | −10 | 60 | 100 | R |
| Step 8 | −10 | 300 | 100 | H |
| Step 9 | −5 | 30 | 100 | R |
| Step 10 | −5 | 300 | 100 | H |
| Step 11 | 0 | 30 | 100 | R |
| Step 12 | 0 | 900 | 100 | H |
| Step 13 | 5 | 180 | 100 | H |
| Step 14 | 15 | 180 | 100 | H |

Secondary Drying

| | | | | |
|---|---|---|---|---|
| Step 1 | 20 | 420 | 100 | H |

R: Ramp;
H: Hold
Total Lyophilization cycle Time: 132.3 Hrs

After lyophilization vacuum was broken and the vials were stoppered, unloaded from lyophilizer, and sealed. The observed moisture content in both batches was 529A: 5.52% w/w and 529B: 7.65% w/w and the observations of the lyophilized samples are shown in Table 24.

TABLE 24
Results of Lyophilization for Batch No 529

| | | | | pH | | Reconstituted |
|---|---|---|---|---|---|---|
| Batch | Appearance of | Reconstitution | Reconstitution | Before | After | Solution |
| No. | Lyophilized Cake | Volume | Time | Lyo | Lyo | Appearance |
| 529 | Dried off white to | 22.0 mL | 90 Sec. | 4.44 | 4.49 | Clear |
| A | Pale Yellow color | | | | | Pale |
| | cake with dark | | | | | Yellow |
| | yellow color at | | | | | color |
| | bottom of cake | | | | | Solution |
| 529 | Dried Pale Yellow | 21.5 mL | 120 Sec. | 4.42 | 4.51 | Clear |
| B | color cake with dark | | | | | Pale |
| | yellow color at | | | | | Yellow |
| | bottom of cake | | | | | color |
| | | | | | | Solution |

The above batches of lyophilization cycle provided acceptable cake appearance, reconstitution time and was further used to produce prototype stability batches. Based on moisture content, the secondary drying was required to be performed at higher temperatures i.e. 25°-30° C. Hence, further lyophilization cycle optimization was carried out for the prototype batch development.

### Preliminary Dilution Study for Compound of Formula (I) Formate Salt as Injection Sample

The lyophilized formulation as prepared above was evaluated for dilution study and 5% Glucose (GS, diluent) solution, sterile water for Injection were used for dilution study. The formulations of batches used were B. No. 529A and 529B.

The lyophilized formulation was reconstituted with sterile water (reconstituting agent) for Injection and the reconstituted formulation was further diluted with different diluents in with compound of Formula (I) formate salt and diluent in a ratio of 1:5 The reconstituted and the diluted samples were evaluated for appearance, pH, and osmolality as shown in Tables 25 and 26.

TABLE 6
Results of reconstituted formulation

| | | | Osmolality of | | |
|---|---|---|---|---|---|
| | | | Reconstituted | pH of | Reconstituted |
| Batch | Reconstitution | Reconstitution | Solution | Reconstituted | Solution |
| No. | Volume | Time | (mOsmol/Kg) | Solution | Appearance |
| 529A | 20.8 mL | 120 Sec. | 714 | 4.63 | Clear Pale |
| | | | | | Yellow colour |
| | | | | | Solution |
| 529B | 21.6 mL | 240 Sec. | 865 | 4.62 | Clear Pale |
| | | | | | Yellow colour |
| | | | | | Solution |

TABLE 7
Results of Dilution Study

| Sr. | | | | | |
|---|---|---|---|---|---|
| No. | Test | 529A | 529B | 529A | 529B |
| 1 | Diluent Used | 5% GS | 5% GS | SWFI | SWFI |
| | | Glucose | Glucose | sterile | sterile |
| | | | | water for | water for |
| | | | | Injection | Injection |
| 2 | Description | * | * | * | * |
| | (Initial) | | | | |
| 3 | pH after dilution | 4.63 | 4.63 | 4.65 | 4.65 |
| | (Initial) | | | | |
| 4 | Osmolality after | 385 | 406 | 146 | 161 |
| | dilutions | (Initial) | (Initial) | (Initial) | (Initial) |
| | (mOsmol/Kg) | | | | |
| 5 | Description | * | * | * | * |
| | (After 6 Hrs) | | | | |

*Clear Slight Pale Yellow Color Solution

The reconstituted solution was clear pale yellow colour solution and was stable up to 6 hrs at room temperature. The product was diluted with 5% glucose solution indicated osmolality between 380-410 mOsmol/Kg, which was in the acceptable range for low volume parenterals. However, samples diluted with sterile water for injection showed hypo-osmolar solutions (140-170 mOsmol/Kg).

### Example 6

### Prototype Stability Batches of Formulations for Injection

The prototype formulation batches for stability studies were prepared as detailed in Table 27 below with batch size of 800 mL each and the lyophilized formulations were prepared as illustrated in Table 28. The stability studies were done for the lyophilized formulations, and the results were recorded as shown in Tables 29 to 32 for the respective batches.

TABLE 27
Composition for Prototype Stability of formulations

| Sr. | | Quantity (mg/mL) | | | |
|---|---|---|---|---|---|
| No. | Material | SF19000586 | SF19000601 | SF19000634 | SF19000649 |
| 1 | Compound of | 20(2%) | 20(2%) | 20(2%) | 20(2%) |
| | Formula(I) | | | | |
| | Formate Salt | | | | |
| 2 | Ascorbic acid | 40(4%) | 40(4%) | 40(4%) | 40(4%) |
| 3 | Lactic acid | 18(1.8% | 18(1.8%) | 18(1.8%) | 18(1.8%) |
| 4 | Niacinamide | 25(2.5%) | 25(2.5%) | 25(2.5%) | 25(2.5%) |
| 5 | L-Arginine | 50(5%) | 30(3%) | 10(1%) | 10(1%) |
| 6 | Mannitol | — | — | — | 20(2%) |
| 7 | Water For | Qs to 1 | Qs to 1 | Qs to 1 | Qs to 1 |
| | Injection | mL | mL | mL | mL |
| 8 | Target pH | pH | pH | pH | pH |
| | | 4.50 ± 0.1 | 4.00 ± 0.1 | 3.50 ± 0.1 | 3.50 ± 0.1 |

TABLE 8
Recipe of the Lyophilization cycle for formulations of prototype
batches of compound formula (1) formate salt drug product

| | | | | |
|---|---|---|---|---|
| Steps | Temp (° C.) | Time (min) | Vacuum (mT) | R/H |

Freezing

| | | | | |
|---|---|---|---|---|
| Step 1 | 5 | 30 | — | R |
| Step 2 | 5 | 60 | — | H |
| Step 3 | −45 | 50 | — | R |
| Step 4 | −45 | 720 | — | H |

Extra Freezing

| | |
|---|---|
| Freeze | −45 |
| Extra Freeze | 30 |
| Condenser | −40 |
| Vacuum | 500 |

Primary Drying

| | | | | |
|---|---|---|---|---|
| Step 1 | −30 | 60 | 200 | R |
| Step 2 | −30 | 1800 | 200 | H |
| Step 3 | −25 | 60 | 200 | R |
| Step 4 | −25 | 1800 | 200 | H |
| Step 5 | −20 | 60 | 100 | R |
| Step 6 | −20 | 900 | 100 | H |
| Step 7 | −10 | 60 | 100 | R |
| Step 8 | −10 | 300 | 100 | H |
| Step 9 | −5 | 30 | 100 | R |
| Step 10 | −5 | 300 | 100 | H |
| Step 11 | 0 | 30 | 100 | R |
| Step 12 | 0 | 900 | 100 | H |
| Step 13 | 5 | 180 | 100 | H |
| Step 14 | 15 | 240 | 100 | H |

Secondary Drying

| | | | | |
|---|---|---|---|---|
| Step 1 | 30 | 480 | 70 | H |

R: Ramp;
H: Hold
Total Lyophilization cycle Time: 134.33 Hrs

TABLE 99
Stability Data of Prototype batch_SF19000586
IUN: Initial Unfiltered Solution; IF: Initial Filtered Solution

| | | SF19000586 | | | | | | | |
|---|---|---|---|---|---|---|---|---|---|
| | | Batch with pH 4.50 ± 0.1 | | | | | | | |
| | | | | Lyophilized samples | | | | | |
| | | Liquid | | | 40° C. ± 2° C./75% | | | | 25° C. ± 2° C./60% |
| | | samples | | | RH ± 5% RH | | | | RH ± 5% RH |
| No. | Test Performed | IUN | IF | Initial | 1 M | 2 M | 3 M | 6 M | 1 M |
| 1 | Description-Lyo | — | — | * | @ | @ | @ | @ | * |
| | cake | | | | | | | | |
| | Description- | ** | ** | ** | @@ | @@ | @@ | @@ | *** |
| | Liquid | | | | | | | | |
| 2 | Reconstitution | — | — | 4.15 min | 15 min | # | Not | Not | 5 min |
| | Time | | | | | | Solubilized | Solubilized | |
| 3 | pH | 4.51 | 4.49 | 4.54 | 4.48 | 4.17 | 4.21 | 4.51 | 4.50 |
| 4 | Water content | — | — | 0.8 | 0.4 | 0.3 | 0.3 | 0.5 | 1.0 |
| | (% w/v) | | | | | | | | |
| 5 | Osmolality | — | — | 739 | 751 | — | 775 | 773 | 740 |
| | (mOsmol/kg) | | | | | | | | |
| 6 | Assay (% w/w) | 100.3 | 102.1 | 104.6 | 82.1 | 53.7 | 44.7 | 29.0 | 97.0 |
| 7 | Related | | | | | | | | |
| | Substances (%) | | | | | | | | |
| | Highest | 0.24 | 0.25 | 0.19 | 17.68 | 28.48 | 33.94 | 44.51 | 1.32 |
| | Unknown | (0.85) | (1.26) | (1.15) | (1.24) | (1.26) | (1.28) | (1.26) | (1.24) |
| | (Relative | | | | | | | | |
| | Retention Time) | | | | | | | | |
| | Total Unknown | 1.17 | 1.21 | 1.07 | 20.2 | 31.32 | 37.17 | 49.25 | 2.27 |
| | Total Impurities | 1.20 | 1.20 | 1.10 | 20.2 | 31.3 | 37.2 | 49.3 | 2.3 |

| | | SF19000586 | | | | | | |
|---|---|---|---|---|---|---|---|---|
| | | Batch with pH 4.50 ± 0.1 | | | | | | |
| | | Lyophilized samples | | | | | | |
| | | 25° C. ± 2° C./60% | | | | | | |
| | | RH ± 5% RH | | | 5° C. ± 3° C. | | | |
| No. | Test Performed | 2 M | 3 M | 6 M | 1 M | 2 M | 3 M | 6 M |
| 1 | Description-Lyo | * | $ | $ | * | * | * | * |
| | cake | | | | | | | |
| | Description- | ** | @@ | @@ | ** | ** | ** | ** |
| | Liquid | | | | | | | |
| 2 | Reconstitution | # | 5 min | 5 min | 5 min | # (1 min) | 3 min | 5 min |
| | Time | (3 min) | | | | | | |
| 3 | pH | 4.25 | 4.32 | 4.51 | 4.47 | 4.27 | 4.34 | 4.53 |
| 4 | Water content | 0.8 | 1.0 | 1.0 | 1.1 | 0.8 | 0.9 | 1.0 |
| | (% w/v) | | | | | | | |
| 5 | Osmolality | — | 749 | 784 | 742 | — | 763 | 780 |
| | (mOsmol/kg) | | | | | | | |
| 6 | Assay (% w/w) | 98.6 | 95.2 | 92.0 | 102.6 | 101.4 | 102.4 | 103.3 |
| 7 | Related | | | | | | | |
| | Substances (%) | | | | | | | |
| | Highest | 1.84 | 3.28 | 8.1 | 0.25 | 0.28 | 0.43 | 0.47 |
| | Unknown | (1.26) | (1.26) | (1.26) | (1.24) | (1.24) | (1.26) | (1.26) |
| | (Relative | | | | | | | |
| | Retention Time) | | | | | | | |
| | Total Unknown | 2.77 | 4.35 | 9.47 | 1.23 | 1.08 | 1.28 | 1.49 |
| | Total Impurities | 2.8 | 4.3 | 9.5 | 1.2 | 1.1 | 1.3 | 1.5 |

* White color cake on top and red color cake at bottom;
** Clear yellow color solution
@ Red yellow colour cake Shrinked completely/Collapsed as a ball;
@@ Yellow Color Suspension;
*** Pale yellow color solution with few grenish to black particles
$: Reddish Yellow color Cake slightly collapse;
#: Kept the sample for 5 min on addition of WFI to wet the cake.
Then shaken vigorously. (Time required to reconstitute the sample)

TABLE 30
Stability Data of Prototype batch _SF19000601

| | | SF19000601 | | | | | | | |
|---|---|---|---|---|---|---|---|---|---|
| | | Batch with pH 4.00 ± 0.1 | | | | | | | |
| | | Liquid | | Lyophilized samples | | | | | |
| Sr. | | samples | | | 40° C. ± 2° C./75% RH ± 5% RH | | | | 25° C.+ 2° C./60% RH ± 5% RH |
| No. | Test Performed | IUN | IF | Initial | 1 M | 2 M | 3 M | 6 M | 1 M |
| 1 | Description-Lyo cake | — | — | * | # | # | # | # | * |
| | Description- | ** | ** | ** | ## | ## | ## | ## | ** |
| | Liquid | | | | | | | | |
| 2 | Reconstitution | — | — | @ | @ | @ | @ | Not | @ |
| | Time | | | (1 min) | (3 min) | (15 min) | (30 min) | Solubilized | (2 min) |
| 3 | pH | 3.95 | 3.94 | 4.01 | 4.00 | 3.97 | 3.86 | 4.00 | 4.01 |
| 4 | Water content | — | — | 1.5 | 0.7 | 1.4 | 0.6 | 0.8 | 1.2 |
| | (% w/v) | | | | | | | | |
| 5 | Osmolality | — | — | 666 | — | — | 637 | 651 | — |
| | (mOsmol/kg) | | | | | | | | |
| 6 | Assay (% w/w) | 101.6 | 100.5 | 100.6 | 88.1 | 83.9 | 63.0 | 32.9 | 99.5 |
| 7 | Related | | | | | | | | |
| | Substances (%) | | | | | | | | |
| | Highest | 0.18 | 0.18 | 0.2 | 6.86 | 13.00 | 18.47 | 27.24 | 0.59 |
| | Unknown | (1.15) | (1.15) | (1.15) | (1.24) | (1.37) | (1.26) | (1.26) | (1.22) |
| | (RRT) | | | | | | | | |
| | Total Unknown | 0.83 | 0.86 | 0.86 | 8.50 | 16.56 | 22.71 | 34.16 | 1.39 |
| | Total Impurities | 0.80 | 0.90 | 0.90 | 8.50 | 16.60 | 22.71 | 34.2 | 1.4 |

| | | SF19000601 | | | | | | |
|---|---|---|---|---|---|---|---|---|
| | | Batch with pH 4.00 ± 0.1 | | | | | | |
| | | Lyophilized samples | | | | | | |
| Sr. | | 25° C. ± 2° C./60% RH ± 5% RH | | | 5° C. ± 3° C. | | | |
| No. | Test Performed | 2 M | 3 M | 6 M | 1 M | 2 M | 3 M | 6 M |
| 1 | Description-Lyo cake | * | $ | $ | * | * | * | * |
| | Description- | ** | ** | ## | ** | ** | ** | ** |
| | Liquid | | | | | | | |
| 2 | Reconstitution | @ | @ | @ | @ | @ | @ | @ |
| | Time | (3 min) | (3 min) | (4 min) | (2 min) | (2 min) | (2 min) | (3 min) |
| 3 | pH | 3.97 | 3.85 | 4.10 | 3.98 | 3.96 | 3.90 | 4.02 |
| 4 | Water content | 1.4 | 1.2 | 1.4 | 1.2 | 1.4 | 1.4 | 1.7 |
| | (% w/v) | | | | | | | |
| 5 | Osmolality | — | 657 | 663 | — | — | 675 | 692 |
| | (mOsmol/kg) | | | | | | | |
| 6 | Assay (% w/w) | 101.2 | 99.9 | 99.3 | 104.1 | 102.4 | 102.5 | 105.2 |
| 7 | Related | | | | | | | |
| | Substances (%) | | | | | | | |
| | Highest | 0.74 | 1.24 | 2.93 | 0.2 | 0.23 | 0.18 | 0.35 |
| | Unknown | (1.37) | (1.26) | (1.26) | (1.15) | (1.27) | (1.26) | (1.26) |
| | (RRT) | | | | | | | |
| | Total Unknown | 1.68 | 2.31 | 4.14 | 0.92 | 0.90 | 1.14 | 1.12 |
| | Total Impurities | 1.7 | 2.31 | 4.1 | 0.9 | 0.9 | 1.14 | 1.1 |

IUN: Initial Unfiltered Solution;
IF: Initial Filtered Solution
* White color cake on top and red color cake at bottom;
** Clear yellow color solution
# Red, yellow colour cake Shrinked completely/Collapsed as a ball
## Yellow to Orange Color Suspension
$ Off-white to pale Yellow colr cake with slightly collapsed structure
@: Kept the sample for 5 min on addition of WFI to wet the cake.
Then shaken vigorously.
(Time in minute required to reconstitute the sample).

TABLE 10
Stability Data of Prototype batch _SF19000634

| | | SF19000634 | | | | | | | |
|---|---|---|---|---|---|---|---|---|---|
| | | Batch with pH 3.50 ± 0.1 (Without Mannitol) | | | | | | | |
| | | | | Lyophilized samples | | | | | |
| | | Liquid | | | 40° C. ± 2° C./75% | | | | 25° C. ± 2° C./60% |
| Sr. | | samples | | | RH ± 5% RH | | | | RH ± 5% RH |
| No. | Test Performed | IUN | IF | Initial | 1 M | 2 M | 3 M | 6 M | 1 M |
| 1 | Description- | — | — | * | ## | ## | ## | ## | * |
| | Lyo cake | | | | | | | | |
| | Description- | ** | ** | ** | *** | *** | *** | *** | ** |
| | Liquid | | | | | | | | |
| 2 | Reconstitution | — | — | 10 Sec | 30 Sec | 30 Sec | 30 Sec | 40 Sec | 30 Sec |
| | Time | | | | | | | | |
| 3 | pH | 3.58 | 3.57 | 3.56 | 3.64 | 3.51 | 3.55 | 3.50 | 3.63 |
| 4 | Water content | — | — | 1.0 | 1.1 | 1 | 1.0 | 1.1 | 1.1 |
| | (% w/v) | | | | | | | | |
| 5 | Osmolality | — | — | 606 | — | — | 567 | 570 | — |
| | (mOsmol/kg) | | | | | | | | |
| 6 | Assay (% w/w) | 98.1 | 100.4 | 103.6 | 95.6 | 95.0 | 92.8 | 87.3 | 99.6 |
| 7 | Related | | | | | | | | |
| | Substances (%) | | | | | | | | |
| | Highest | 0.18 | 0.18 | 0.2 | 2.08 | 3.66 | 5.84 | 9.74 | 0.38 |
| | Unknown | (1.15) | (1.15) | (1.15) | (1.24) | (1.24) | (1.26) | (1.26) | (1.15) |
| | Total Unknown | 0.87 | 0.87 | 0.93 | 4.50 | 6.81 | 11.02 | 17.91 | 1.15 |
| | Total Impurities | 0.90 | 0.90 | 0.90 | 4.5 | 6.8 | 11.0 | 17.9 | 1.1 |

| | | SF19000634 | | | | | | |
|---|---|---|---|---|---|---|---|---|
| | | Batch with pH 3.50 ± 0.1 (Without Mannitol) | | | | | | |
| | | Lyophilized samples | | | | | | |
| | | 25° C. ± 2° C./60% | | | | | | |
| Sr. | | RH ± 5% RH | | | 5° C. ± 3° C. | | | |
| No. | Test Performed | 2 M | 3 M | 6 M | 1 M | 2 M | 3 M | 6 M |
| 1 | Description- | * | $ | $ | * | * | * | * |
| | Lyo cake | | | | | | | |
| | Description- | ** | ** | ** | ** | ** | ** | ** |
| | Liquid | | | | | | | |
| 2 | Reconstitution | 20 Sec | 20 Sec | 30 Sec | 30 Sec | 20 Sec | 20 Sec | 30 Sec |
| | Time | | | | | | | |
| 3 | pH | 3.51 | 3.52 | 3.57 | 3.62 | 3.49 | 3.5 | 3.61 |
| 4 | Water content | 1.3 | 1.1 | 1.1 | — | 1.3 | 1.1 | 1.0 |
| | (% w/v) | | | | | | | |
| 5 | Osmolality | — | 579 | 588 | — | — | 598 | 634 |
| | (mOsmol/kg) | | | | | | | |
| 6 | Assay (% w/w) | 100.6 | 103.6 | 105.7 | 103.0 | 101.8 | 106.0 | 108.9 |
| 7 | Related | | | | | | | |
| | Substances (%) | | | | | | | |
| | Highest | 0.53 | 0.6 | 1.02 | 0.2 | 0.25 | 0.19 | 0.20 |
| | Unknown | (1.15) | (1.16) | (1.16) | (1.15) | (1.15) | (1.16) | (1.16) |
| | Total Unknown | 1.31 | 2.06 | 2.36 | 0.91 | 1.32 | 1.08 | 1.04 |
| | Total Impurities | 1.3 | 2.06 | 2.4 | 0.9 | 1.3 | 1.08 | 1.0 |

IUN: Initial Unfiltered Solution;
IF: Initial Filtered Solution
* Off white to Pale Yellow color cake;
** Clear Slight Pale yellow color solution
## Off white to light red color cake with slight collapse;
*** Yellow to orange color suspension
$ Off-white to pale Yellow colr cake with slightly collapsed structure

TABLE 11
Stability Data of Prototype batch_SF19000649

| | | SF19000649 | | | | | | | |
|---|---|---|---|---|---|---|---|---|---|
| | | Batch with pH 3.50 ± 0.1 (With Mannitol) | | | | | | | |
| | | | | Lyophilized samples | | | | | |
| | | Liquid | | | 40° C. ± 2° C./75% | | | | 25° C. ± 2° C./60% |
| Ser. | Test | samples | | | RH ± 5% RH | | | | RH ± 5% RH |
| No. | Performed | IUN | IF | Initial | 1 M | 2 M | 3 M | 6 M | 1 M |
| 1 | Description- | — | — | # | # | ## | ## | ## | # |
| | Lyo cake | | | | | | | | |
| | Description- | * | * | * | *** | *** | *** | *** | * |
| | Liquid | | | | | | | | |
| 2 | Reconstitution | — | — | 15 Sec | 30 sec | 2 min | 3 min | >10 min | <10 Sec |
| | Time | | | | | | | | |
| 3 | pH | 3.55 | 3.56 | 3.53 | 3.49 | 3.46 | 3.56 | 3.83 | 3.47 |
| 4 | Water content | — | — | 0.8 | 1.1 | 1.1 | 1.2 | 1.2 | 1.1 |
| | (% w/v) | | | | | | | | |
| 5 | Osmolality | — | — | 739 | — | — | 694 | — | — |
| | (mOsmol/kg) | | | | | | | | |
| 6 | Assay (% | 104.9 | 105.0 | 108.9 | 98.7 | 97.3 | 90.8 | 77.7 | 102.6 |
| | w/w) | | | | | | | | |
| 7 | Related | | | | | | | | |
| | Substances | | | | | | | | |
| | (%) | | | | | | | | |
| | Highest | 0.24 | 0.24 | 0.24 | 2.15 | 4.51 | 5.97 | 11.442 | 0.41 |
| | Unknown | | | | | | | | |
| | | (1.15) | (1.15) | (1.15) | (1.24) | (1.26) | (1.26) | (1.25) | (1.15) |
| | Total | 0.41 | 0.41 | 0.43 | 3.86 | 8.99 | 10.58 | 22.2 | 0.70 |
| | Unknown | | | | | | | | |
| | Total | 0.40 | 0.40 | 0.40 | 3.9 | 9.0 | 10.58 | 22.2 | 0.70 |
| | Impurities | | | | | | | | |

| | | SF19000649 | | | | | | |
|---|---|---|---|---|---|---|---|---|
| | | Batch with pH 3.50 ± 0.1 (With Mannitol) | | | | | | |
| | | Lyophilized samples | | | | | | |
| | | 25° C. ± 2° C./60% | | | | | | |
| Ser. | Test | RH ± 5% RH | | | 5° C. ± 3° C. | | | |
| No. | Performed | 2 M | 3 M | 6 M | 1 M | 2 M | 3 M | 6 M |
| 1 | Description- | # | ## | ### | # | # | # | # |
| | Lyo cake | | | | | | | |
| | Description- | * | * | ** | * | * | * | * |
| | Liquid | | | | | | | |
| 2 | Reconstitution | <10 Sec | <10 Sec | 1 Min | <10 Sec | <10 Sec | <10 Sec | 1 Min |
| | Time | | | 45 Sec | | | | 15 Sec |
| 3 | pH | 3.43 | 3.54 | 3.85 | 3.48 | 3.45 | 3.53 | 3.83 |
| 4 | Water content | 1.1 | 1.1 | 1.1 | 1.0 | 1.0 | 1.1 | 1.1 |
| | (% w/v) | | | | | | | |
| 5 | Osmolality | — | 691 | — | — | — | 710 | — |
| | (mOsmol/kg) | | | | | | | |
| 6 | Assay (% | 106.6 | 102.3 | 102.4 | 105.1 | 106.9 | 104.4 | 103.7 |
| | w/w) | | | | | | | |
| 7 | Related | | | | | | | |
| | Substances | | | | | | | |
| | (%) | | | | | | | |
| | Highest | 0.58 | 0.68 | 1.093 | 0.25 | 0.26 | 0.24 | 0.305 |
| | Unknown | | | | | | | |
| | | (1.16) | (1.7) | (1.16) | (1.15) | (1.16) | (1.17) | (1.16) |
| | Total | 1.05 | 1.47 | 1.7 | 0.42 | 0.53 | 0.44 | 0.41 |
| | Unknown | | | | | | | |
| | Total | 1.1 | 1.47 | 1.7 | 0.4 | 0.5 | 0.44 | 0.41 |
| | Impurities | | | | | | | |

IUN: Initial Unfiltered Solution;
IF: Initial Filtered Solution
# Off white to Pale Yellow color cake;
* Clear Slight Pale yellow color solution;
## Off white to Pale Yellow color cake with collapse/meltback;
** Clear Yellow color solution
### Pale yellow to red color cake;
*** Yellow color suspension
R: Ramp;
H: Hold
Total Lyophilization cycle Time: 97.08 Hrs

All the batches of formulations of the compound of Formula (I) formate salt were shown to meltback/collapse of cake at 40° C.±2° C./75% RH±5% RH after 6 months, and at 25° C.±2° C./60% RH±5% RH slight collapsed cake structure with change in appearance was observed in all batches compared to initial. In all batches lyophilized cake after 6 months at 40° C.±2° C./75% RH±5% RH and 25° C.±2° C./60% RH±5% RH, showed difficulties in reconstitutions, yielding yellow to orange colour suspension.

The stability of lyophilized formulations at 2°-8° C. after 6 months (all batches) indicated similar cake structure as that of initial cake and there was no difficulty in reconstitution of lyophilized formulation. All batches of formulations showed no change in pH of formulation stored at all stability conditions. Decrease in assay and increase in impurities was observed for the formulations stored at 40° C.±2° C./75% RH±5% RH and 25° C.±2° C./60% RH±5% RH, whereas no significant difference was observed in assay and impurity profile for the formulation's batches stored at 2°-8° C. There was no significant impact of mannitol on stabilization of the drug product.

Hence, the lyophilized formulation of the compound of Formula(I) formate salt for injection was evaluated at different pH conditions i.e., at pH 3.50, 4.00 & 4.50 and the storage condition of 2°-8° C. selected for the pH range studies. The developed formulations were further slightly modified for the process optimization.

### Process Optimization Study of Formulation for Injection

The compound of Formula (I) formate salt was not solubilized at room temperature even after stirring for 3 hrs but the heating up to 40° C.-50° C. solubilized the compound of Formula (I) in a formulation with vehicle. This resulted in significant impact of temperature and stirring time on related substances of the formulation in unfiltered sample and filtered samples specially sample manufactured at 50° C. with 3 hrs stirring. Hence the process was optimized further to solubilize the compound of Formula (I) formate salt at lower temperature.

### Effect of Initial Volume of WFI on Solubilization of Compound of Formula (I) Formate Salt

The formulations of the present disclosure were prepared by varying the processing temperature of the preparation of the formulation and its effect on solubilization of compound of Formula (I) formate salt were studied. A batch size of 50 ml each was used.

TABLE 12
Impact of processing temperature on solubilization
of compound of Formula (I) formate salt

| | | Quantity (mg/mL) | | | |
|---|---|---|---|---|---|
| | | 777A | | 777B | |
| Sr. | | Processing Temperature | | | |
| No. | Ingredients | 25° C. | | 45° C. | |
| 1 | Compound of formula (I) | 20 | (2%) | 20 | (2%) |
| | formate salt | | | | |
| 2 | Ascorbic Acid | 40 | (4%) | 40 | (4%) |
| 3 | Lactic acid 90% | 18 | (1.8%) | 18 | (1.8%) |
| 4 | Niacinamide | 25 | (2.5%) | 25 | (2.5%) |
| 5 | L-Arginine | 50 | (5%) | 50 | (5%) |

| | | | |
|---|---|---|---|
| 6 | Water For Injection (Qs to) | 1 mL | 1 mL |
| 7 | Target pH | 4.50 | 4.50 |

% expressed in w/v

The ascorbic acid and lactic acid were dissolved in 20% of Water for Injection (25° C.-30° C.) of total batch size. Then, compound of Formula (I) formate salt was added in the above solution and kept for stirring at respective temperature condition as shown in Table 33. Niacinamide was added to the above solution at room temperature (RT) and dissolved by stirring. L-Arginine was added to the above solution and dissolved by stirring. The volume was made up to 50 mL with WFI and kept for stirring at RT. The samples were kept for physical observation at RT.

TABLE 13
Observation of process optimization on
solubilization of compound formula (I)

Observations

| | |
|---|---|
| 777A | 777B |

| | |
|---|---|
| Sr. | Processing Temperature |

| No. | Test | 25° C. | 45° C. |
|---|---|---|---|
| 1 | Appearance (Initial) | * | * |
| 2 | pH (Initial) | 4.63 | 4.66 |
| 3 | Appearance on standing | ** | ** |
| | | (18 Hrs) | (18 Hrs) |

*Clear Pale Yellow color Solution
**Clear Pale Yellow color Solution with dust like particles

From Table 34 above it can be understood that the compound of Formula(I) formate salt could be solubilized at 25° C. (room temperature, RT) and at 45° C. using a smaller amount of WFI at the initial stage of the manufacturing process i.e., 20% of total batch size.

The solution manufactured using this process remained stable at RT (5 to 18 Hrs) and indicated that the heating step in the manufacturing process could be eliminated while solubilizing the compound of formula(I) formate in the given formulation of vehicle as illustrated herein.

To determine glass transition temperature, differential scanning calorimetry (DSC) thermogram analysis was undertaken. In the DSC thermogram, glass transition temperature was not observed. In both the thermograms exothermic peak at −20° C. to −25° C. was observed; this was due to water crystallization in the sample and endothermic peak at 0° C. indicates ice melting phenomenon.

During cooling (second time) to −70° C., a peak was observed at −32° C. to −36° C. However, this peak could be due to recrystallization of frozen matrix. Using DSC analysis, detecting the glass transition temperature of the formulation was challenging, this was because the formulation composed acids and different salts which probably had very lower glass transition temperature. Further investigation was required to identify the glass transition temperature or collapse temperature.

### Lyophilization Cycle Development for the Formulations

Based on the lyophilization formulations developed herein, further optimization was done. Annealing step was incorporated during freezing stage to form larger ice crystals. The preparation process is as follows i.e., solubilizing the compound of Formula(I) formate salt at room temperature and further development of the lyophilization cycle for higher solid content.

TABLE 35
Formulation for lyophilization cycle development
of compound of formula (I) formate salt

| | | Weight | Quantity |
|---|---|---|---|
| | | % | (mg/mL) |
| Sr. No. | Ingredients | (% w/v) | SF19000829 |
| 1 | Compound of Formula (I) formate | 2 | 20 |
| | salt | | |
| 2 | Ascorbic Acid | 4 | 40 |
| 3 | Lactic acid 90% | 1.8 | 18 |
| 4 | Niacinamide | 2.5 | 25 |
| 5 | L-Arginine | 5 | 50 |
| 6 | Water For Injection (Qs to) | Qs | 1 mL |
| 7 | Target pH | 4.5 | 4.50 |

The formulation as shown in Table 35 was prepared by the process defined herein. Ascorbic acid (solubilizing agent) was dissolved in Water for Injection (second solvent, ˜20% of batch size, 25° C.-30° C.) followed by addition of lactic acid (solubilizing agent) under stirring for 5 mins. Then, the compound of Formula (I) as formate salt was added to the above solution and dissolved by stirring for 3 hrs at room temperature to obtain a first solution. To the first solution, niacinamide (hydrotropic agent) was then added and was dissolved for 10 min to obtain a second solution, followed by addition of L-Arginine (pH modifier) under stirring for 10 min to obtain the formulation. The volume of formulation was made up to batch size using WFI (25°-30° C.) and mixed for 15 min. The solution was filtered through 0.2μ PES filter (47 mm) using vacuum filtration assembly. A batch size of 300 mL of each formulation was prepared.

Table 36 below shows the process followed for the lyophilization cycle of compound of Formula (I) formate salt. The formulation was subjected to freezing at a temperature range of −50 to 0° C., preferably in a range of −45 to 0° C., sequentially as shown in Table 36 below. Post freezing, the formulation was subjected to additional freezing and drying to obtain the lyophilized sample. The drying was carried out at a pressure range of 10μ bar to 1 bar. After lyophilization vacuum was broken with nitrogen gas. Vials were stoppered, unloaded from lyophilizer, and sealed. The lyophilized formulation was analysed for its appearance and the characteristics as shown in Table 37.

TABLE 36
Lyophilization cycle for compound of Formula
(I) formate salt_Batch No SF19000829

| | | | | |
|---|---|---|---|---|
| Steps | Temp (° C.) | Time (min) | Vacuum (mT) | R/H |

Freezing

| | | | | |
|---|---|---|---|---|
| Step 1 | 0 | 30 | — | R |
| Step 2 | 0 | 60 | — | H |
| Step 3 | −20 | 30 | — | R |
| Step 4 | −20 | 120 | — | H |
| Step 5 | −45 | 60 | — | R |
| Step 6 | −45 | 240 | — | H |
| Step 7 | −20 | 30 | — | R |
| Step 8 | −20 | 180 | — | H |
| Step 9 | −45 | 30 | — | R |
| Step 10 | −45 | 600 | — | H |

Extra Freezing

| | |
|---|---|
| Freeze | −45 |
| Extra Freeze | 30 |
| Condenser | −40 |
| Vacuum | 500 |

Primary Drying

| | | | | |
|---|---|---|---|---|
| Step 1 | −30 | 20 | 250 | R |
| Step 2 | −30 | 1200 | 250 | H |
| Step 3 | −25 | 60 | 200 | R |
| Step 4 | −25 | 600 | 200 | H |
| Step 5 | −20 | 30 | 100 | R |
| Step 6 | −20 | 720 | 100 | H |
| Step 7 | −10 | 30 | 100 | R |
| Step 8 | −10 | 300 | 100 | H |
| Step 9 | −5 | 30 | 100 | R |
| Step 10 | −5 | 180 | 100 | H |
| Step 11 | 0 | 30 | 100 | R |
| Step 12 | 0 | 705 | 100 | H |
| Step 13 | 5 | 15 | 100 | R |
| Step 14 | 5 | 60 | 100 | H |
| Step 15 | 15 | 15 | 100 | R |
| Step 16 | 15 | 90 | 100 | H |

Secondary Drying

| | | | | |
|---|---|---|---|---|
| Step 1 | 30 | 360 | 70 | H |

R: Ramp;
H: Hold
Total Lyophilization cycle Time: 97.08 Hrs

TABLE 37
Results of Lyophilized formulation of compound of Formula(I)
formate salt drug product_Batch No SF19000829

| | | | | pH | | Reconstituted |
|---|---|---|---|---|---|---|
| Batch | Appearance of | Reconstitution | Reconstitution | Before | After | Solution |
| No. | Lyophilized Cake | Volume | Time | Lyo | Lyo | Appearance |
| SF19000829 | Dried off white to | 22 mL | 2 min | 4.59 | 4.51 | Clear |
| | Pale Yellow colour | | | | | Pale |
| | cake with dark | | | | | Yellow |
| | yellow colour at | | | | | colour |
| | bottom of cake. | | | | | Solution |
| | No Shrinkage of | | | | | |
| | cake | | | | | |

The annealing step during freezing aided to improve the cake structure providing intact cake. The lyophilized cake was easily reconstituted with water for injection within 2 minutes forming clear pale yellow colour solution. No change in the pH/shift in pH of the reconstituted solution was observed due to lyophilization. This lyophilization cycle was then modified slightly at the end of primary drying step to target the lyophilized cycle of 96 hrs which provided similar lyophilized cake structure, reconstitution time and same lyophilization cycle was adopted for further development with higher solid content in lyophilized cake.

### Diluent Compatibility Study of the Formulations

The diluent compatibility study was performed with 5% Dextrose Injection as 1.0 mg/mL, 3.33 mg/mL and 10.0 mg/mL strength after dilution.

TABLE 38

| Sr. | | Quantity | Quantity for Batch (g) | |
|---|---|---|---|---|
| No. | Ingredients | (mg/mL) | 382 | 475 |
| 1 | Formula (I) (as formate salt) | 20 | 3.108* | 2.279* |
| 2 | Ascorbic acid | 40 | 6.00 | 4.40 |
| 3 | Lactic acid | 18 | 2.70 | 0.198 |
| 4 | Niacinamide | 25 | 3.75 | 2.75 |
| 5 | L-Arginine | 50 | 7.50 | 5.50 |
| 6 | Water For Injection | Qs to 1 | Qs to 150 | Qs to 110 |
| | | mL | mL | |

The lyophilized formulation as shown in Table 38 was prepared as per the process illustrated below. The Water for Injection (WFI) was purged with nitrogen gas for 1 hr. 20% of total batch size of nitrogen purged Water for Injection (RT) was collected in glass beaker. To this, weighed quantity of ascorbic acid was added and kept under stirring for 5 min. at RT, using magnetic stirrer. The solution appeared clear and was a light pale yellow colour solution. Weighed quantity of lactic acid was added to the above solution under stirring at RT. The solution appeared clear and was still a light pale yellow colour solution. Weighed quantity of compound of Formula(I) formate salt was added in above solution and kept under stirring for 120 min at RT. The solution now became translucent and was a dark yellow colour solution with few dust like particles. 40% of total batch size of Nitrogen purged WFI was added to the above solution and kept under stirring for 10 min at RT. Translucent, yellow colour solution with some dust like particles was observed. Weighed quantity of Niacinamide was added to the above and was kept under stirring for 15 min at RT. Translucent, dark yellow colour solution with some dust like particles was observed. Weighed quantity of L-Arginine was added to the above and kept under stirring for 15 min at RT. The solution became translucent, dark yellow colour solution with some dust like particles. Volume of above step solution was made up to required batch size by using nitrogen purged WFI (RT) and stirred for 15 min at RT. (Appearance: Clear to Translucent, dark yellow colour solution with some dust (external) like particles). Above solution was filtered through 0.2 PES (Polyether sulfone) membrane filter Appearance (after filtration) was a clear, yellow colour solution. The filtered solution was stored at 5°±3° C. for ˜18 hours till further use.

The above filtered bulk solution was thawed to room temperature from 5°±3° C. for 1 hr. The solution appeared clear, dark yellow colour solution, and free of any visible particles. The details of diluent and IV (intravenous) set are shown in Table 39 below. Table 40 shows details of dilution stability and IV set compatibility of Formula (I) formate salt for Injection with different concentrations and Table 41 shows Chemical Analysis and particulate matter of the formulation.

TABLE 39
Details of diluent and IV set.

| Sr. | |
|---|---|
| No. | Details |
| 1 | Diluent Bag: |
| | 5% Glucose Injection, 250 mL |
| 2 | IV tubing: |
| | Infusion set, light resistant, 15 μ filter |
| 3 | Needle: |
| | BD Insyte 20 GA 1.00 IN; 1.1 × 25 mm |
| 4 | Connector: |
| | Luer Access Device |

TABLE 40
Dilution stability and IV set compatibility of Formula(I) formate salt for Injection
with different concentrations- Chemical Analysis and particulate matter

| | | Volume of | | | | | | | |
|---|---|---|---|---|---|---|---|---|---|
| | | 5% Glucose | | | | | | Final | |
| | | to be withdrawn | Volume of | | | | | concentration | |
| | | and discarded from | Formula (I) | | | | | of compound | |
| | | infusion bag of | for injection | | Volume of Diluent | | Total | of formula(I) | |
| Sr. No. | Diluent | 250 mL pack size | (20 mg/ mL) | | to be taken | | volume | formate salt per mL | |
| SF21000382A | 5% | 137.5 mL | 7.5 | mL | 112.5 | mL | 120 mL | 1.25 | mg/mL |
| | Glucose | | | | | | | | |
| SF21000382B | 5% | 145.0 mL | 15.0 | mL | 105.0 | mL | 120 mL | 2.5 | mg/mL |
| | Glucose | | | | | | | | |
| SF21000382C | 5% | 151.0 mL | 21.0 | mL | 99.0 | mL | 120 mL | 3.5 | mg/mL |
| | Glucose | | | | | | | | |
| SF21000382D | 5% | 154.0 mL | 24.0 | mL | 96.0 | mL | 120 mL | 4.0 | mg/mL |
| | Glucose | | | | | | | | |
| SF21000382E | 5% | 160.0 mL | 30.0 | mL | 90.0 | mL | 120 mL | 5.0 | mg/mL |
| | Glucose | | | | | | | | |

TABLE 41
Results of In-use stability study (Chemical Analysis)

| | | | Compound of Formula (I) (formate salt) | | | | |
|---|---|---|---|---|---|---|---|
| | | | strength in diluent bags and IV set | | | | |
| | | | A | B | C | D | E |
| | | | (1.25 | (2.50 | (3.50 | (4.0 | (5.0 |
| Test Parameter | Specifications | Time | mg/mL) | mg/mL) | mg/mL) | mg/mL) | mg/mL) |
| Description | clear, | Initial | * | * | * | * | * |
| | colorless | | | | | | |
| | to pale | 2 Hrs. | * | * | * | * | * |
| | yellow | 4 Hrs. | * | * | * | * | * |
| | color | 6 Hrs. | * | * | | * | * |
| | solution | 8 Hrs. | * | * | * | * | * |
| pH of diluted | Report | Initial | 4.59 | 4.54 | 4.54 | 4.55 | 4.54 |
| solution | results | 2 Hrs. | 4.55 | 4.54 | 4.55 | 4.54 | 4.54 |
| | | 4 Hrs. | 4.58 | 4.57 | 4.55 | 4.55 | 4.54 |
| | | 6 Hrs. | 4.58 | 4.56 | 4.56 | 4.55 | 4.54 |
| | | 8 Hrs. | 4.58 | 4.56 | 4.55 | 4.55 | 4.53 |
| Osmolality | Report | Initial | 343 | 376 | 404 | 421 | 449 |
| (mOsmol/Kg) | results | 2 Hrs. | 343 | 382 | 406 | 420 | 448 |
| | | 4 Hrs. | 345 | 380 | 414 | 423 | 451 |
| | | 6 Hrs. | 342 | 380 | 416 | 434 | 457 |
| | | 8 Hrs. | 341 | 377 | 410 | 435 | 448 |
| Assay of | NLT 90.0% | Initial | 103.0 | 98.6 | 98.3 | 98.4 | 97.6 |
| Formula (I) | and | | | | | | |
| as formate | NMT 110.0% | 2 Hrs. | 103.1 | 99.3 | 99.3 | 98.7 | 97.8 |
| salt (%) | of | | | | | | |
| | the labelled | 4 Hrs. | 103.7 | 99.6 | 99.5 | 98.9 | 97.5 |

TABLE 42
Results of In-use stability study (Impurity Analysis)

| | | | | Compound of Formula (I) | | | | |
|---|---|---|---|---|---|---|---|---|
| | | | | (formate salt) strength in | | | | |
| | | | | diluent bags and IV set | | | | |
| | | | | A | B | C | D | E |
| | Test | | | (1.25 | (2.50 | (3.50 | (4.0 | (5.0 |
| Specifications | Para [symbol] | | Time | mg/mL) | mg/mL) | mg/mL) | mg/mL) | mg/mL) |
| Amount of | | | 6 | 103.1 | 99.1 | 99.1 | 98.6 | 98.0 |
| Formula (I) | | | Hrs. | | | | | |
| (formate salt) | | | 8 | 103.1 | 99.7 | 98.0 | 98.3 | 97.1 |
| | | | [symbol] | | | | | |
| Report results | Related | Highest | Initial | 0.409 | 0.420 | 0.437 | 0.424 | 0.444 |
| | Substances | Unknown | | | | | | |
| | (%)or | (RRT) | | (0.8 [symbol] | (0.8 [symbol] | (0.8 [symbol] | (0.8 [symbol] | (0.8 [symbol] |
| | impurities | Total | | 1.6 | 1.6 | 1.6 | 1.7 | 1.7 |
| | | [symbol] | | | | | | |
| Report results | | Highest | 2 | 0.427 | 0.412 | 0.425 | 0.460 | 0.444 |
| | | Unknown | Hrs. | | | | | |
| | | (RRT) | | (0.8 [symbol] | (0.8 [symbol] | (0.8 [symbol] | (0.8 [symbol] | (0.8 [symbol] |
| | | Total | | 1.8 | 1.7 | 1.8 | 2.0 | 1.8 |
| | | [symbol] | | | | | | |
| Report results | | Highest | 4 | 0.412 | 0.465 | 0.430 | 0.422 | 0.420 |
| | | Unknown | Hrs. | | | | | |
| | | (RRT) | | (0.8 [symbol] | (0.8 [symbol] | (0.8 [symbol] | (0.8 [symbol] | (0.8 [symbol] |
| | | Total | | 1.8 | 1.8 | 1.8 | 1.8 | 1.8 |
| | | [symbol] | | | | | | |
| Report results | | Highest | 6 | 0.435 | 0.538 | 0.658 | 0.828 | 0.864 |
| | | Unknown | Hrs. | | | | | |
| | | (RRT) | | (1.2 [symbol] | (1.2 [symbol] | (1.2 [symbol] | (1.2 [symbol] | (1.2 [symbol] |
| | | Total | | 2.0 | 2.2 | 2.3 | 2.5 | 2.6 |
| | | [symbol] | | | | | | |
| Report results | | Highest | 8 | 0.954 | 1.334 | 1.334 | 0.683 | 1.258 |
| | | Unknown | Hrs. | | | | | |
| | | (RRT) | | (1.2 [symbol] | (1.2 [symbol] | (1.2 [symbol] | (1.2 [symbol] | (1.2 [symbol] |
| | | Total | | 2.8 | 3.1 | 3.0 | 2.5 | 2.9 |
| | | [symbol] | | | | | | |

*Clear slightly Pale Yellow color solution free from visible particles; [symbol]
indicates data missing or illegible when filed

TABLE 43
Results of In-use stability study (Particulate matter

| | | | | Compound of Formula(I) formate salt | | | | | |
|---|---|---|---|---|---|---|---|---|---|
| | | | | strength in diluent bags and IV set | | | | | |
| | | | | A | B | C | D | E | P |
| Test | Proposed | | Time | (1.25 | (2.50 | (3.50 | (4.0 | (5.0 | (5.0 |
| Parameter | Specifications | | Points | mg/mL) | mg/mL) | mg/mL) | mg/mL) | mg/mL) | mg/mL) |
| Particulate | a) Visible - | | Initial | * | * | * | * | * | # |
| matter | Visual test: Should | | | | | | | | |
| | be free form visible | | 4 Hrs. | * | * | * | * | * | # |
| | particles | | 8 Hrs. | * | * | * | * | * | # |
| | b) Sub- | ≥10 | Initial | 6 | 2 | 4 | 3.5 | 17 | 28@ |
| | Visible | μm: | | | | | | | |
| | (By | NMT 25 | | | | | | | |
| | light | per mL | | | | | | | |
| | obscuration | ≥25 | | 0 | 0 | 0 | 0 | 0 | 0 |
| | method): | μm: | | | | | | | |
| | | NMT 3 | | | | | | | |
| | | per mL | | | | | | | |
| | | ≥10 | 4 Hrs. | 1 | 0.5 | 1.5 | 3.0 | 1.5 | 1.5 |
| | | μm: | | | | | | | |
| | | NMT 25 | | | | | | | |
| | | per mL | | | | | | | |
| | | ≥25 | | 0 | 0 | 0 | 0 | 0 | 0 |
| | | μm: | | | | | | | |
| | | NMT 3 | | | | | | | |
| | | [symbol] | | | | | | | |
| | | ≥10 | 8 Hrs. | 3 | 3.5 | 1.5 | 0 | 1.5 | 1.0 |
| | | μm: | | | | | | | |
| | | NMT 25 | | | | | | | |
| | | [symbol] | | | | | | | |
| | | ≥25 | | 0 | 0 | 0 | 0 | 0 | 0 |
| | | μm: | | | | | | | |
| | | NMT 3 | | | | | | | |
| | | [symbol] | | | | | | | |

* Clear slightly pale-yellow color solution free from visible particles;
# Clear colorless to slightly pale-yellow color solution free from visible particles
@Placebo at Initial time point was on higher side might be due to vial contamination. [symbol]
indicates data missing or illegible when filed

The formulation comprising the compound of Formula (I) formate salt for Injection (Bulk solution) was diluted with 5% Glucose injection in different concentration. The diluted solutions were held at room temperature (20°-30° C.) along with IV sets. These diluted solutions were evaluated for different physicochemical parameters like description, pH, osmolality, assay of compound of Formula(I) formate salt, related substances, and particulate matter.

The diluted solution was found to be a clear, slightly pale-yellow color solution, free from visible particles and remained stable up to 8 hrs at room temperature (20°-30° C.). The diluted solution indicated a pH of 4.50±0.20 and remained stable up to 8 hrs. The diluted solution was iso-osmolar in nature at concentration of 1.25 mg/mL (270 to 330 mOsmol/kg) as shown in Table 41 and remained stable up to 24 hrs. The remaining concentration diluted solution (2.50, 3.50, 4.0 and 5.0 mg/mL) were slightly hyperosmolar solution (370 to 460 mOsmol/kg), however this osmolality range solutions could be administered as large volume parenteral solutions. The diluted solution remained stable up to 8 hrs, no significant change was observed in assay of compound of Formula (I) formate salt. There was increase in impurity levels was observed from initial (0 Hrs) to 8 Hrs hold duration, however impurity levels were within the specified limits of bulk solution of formulation of compound of Formula (I) formate salt for Injection (Total impurity: NMT 4%, Table 42). The particulate matter in diluted solution complies as per limit mentioned for large volume parenteral in chapter USP <788> Particulate Matter in Injections. The particulate matter (Table 43) at initial (0 hrs) for placebo was not complying as per limits. This might be due to contamination that occurred during sample handling/sample analysis.

Based on In-use stability study of formulation of compound of Formula (I) for Injection (as formate salt bulk solution), it was recommended that, the bulk solution of compound of Formula (I) formate salt for Injection should be further diluted with 5% Glucose Injection (freeflex bags) and should be used within 6-8 hrs after its dilution along with proposed IV set when stored at room temperature (20°-30° C.) and protected from light. The following concentrations of formulation of compound of Formula (I) formate salt in diluted solution with 5% Glucose Injection were recommended for its administration are 1.25 mg/mL, 2.50 mg/mL, 3.50 mg/mL, 4.0 mg/mL, and 5.0 mg/mL.

The respective placebo solution of different strengths also should be used within 6 hrs after its dilution along with proposed IV set when stored at room temperature (20°-30° C.) and protected from light.

### Stability Study of the Formulations

Lead batches stability study for formulations of compound of Formula (I) formate salt for Injection with 500 mg/vial and a batch size of 1600 mL was prepared using with the ingredients mentioned in Table 44.

TABLE 44
Formulations of compound of Formula (I) formate
salt for stability studies SF20000011

| Sr. | | | |
|---|---|---|---|
| No. | Ingredients | Quantity (mg/mL) | |
| 1 | Compound of Formula (I) formate | 20 | (2%) |
| | salt | | |
| 2 | Ascorbic Acid | 40 | (4%) |
| 3 | Lactic Acid | 18 | (1.8%) |
| 4 | Niacinamide | 25 | (2.5%) |
| 5 | L-Arginine | 50 | (5%) |

| | | |
|---|---|---|
| 6 | Water For Injection (Qs to) | 1 mL |
| 7 | Target pH | 4.50 ± 0.1 |

Formulation was prepared by the process as described herein. Ascorbic acid was dissolved in water for injection (20% of batch size, 25° C.-30° C.) and was dissolved by stirring for 30 min followed by addition of lactic acid under stirring for 15 min at RT, and compound of Formula (I) formate salt under stirring for 150 min at RT to obtain the first solution. To the first solution, niacinamide was added and dissolved under stirring for 30 min. at RT to obtain a second solution. To the second solution added additional 40% of WFI (of total batch size) under stirring for 45 min and L-arginine (pH modifier) was added under stirring for 30 min at RT to obtain the formulation. WFI was added to makeup to the volume of formulation solution to batch 5 size. Then the solution was filtered through 0.2μ PBS filter (47 mm, Lot No. 071715407, Sartorius) using vacuum filtration assembly to obtain the formulation. The 25.7 mL of filtered solution was filled in 50 mL clear USP type I Tubular vials, half stopper the vial and was loaded for lyophilization. The lyophilization cycle used for the formulation is as below.

TABLE 45
Recipe of the Lyophilization cycle
of formulation _Batch SF20000011

| | | | | |
|---|---|---|---|---|
| Steps | Temp (° C.) | Time (min) | Vacuum (mT) | R/H |

Freezing

| | | | | |
|---|---|---|---|---|
| Step 1 | 0 | 30 | — | R |
| Step 2 | 0 | 60 | — | H |
| Step 3 | −20 | 30 | — | R |
| Step 4 | −20 | 120 | — | H |
| Step 5 | −45 | 60 | — | R |
| Step 6 | −45 | 240 | — | H |
| Step 7 | −20 | 30 | — | R |
| Step 8 | −20 | 180 | — | H |
| Step 9 | −45 | 30 | — | R |
| Step 10 | −45 | 600 | — | H |

Extra Freezing

| | |
|---|---|
| Freeze | −45 |
| Extra Freeze | 30 |
| Condenser | −40 |
| Vacuum | 500 |

Primary Drying

| | | | | |
|---|---|---|---|---|
| Step 1 | −30 | 20 | 250 | R |
| Step 2 | −30 | 1200 | 250 | H |
| Step 3 | −25 | 30 | 200 | R |
| Step 4 | −25 | 600 | 200 | H |
| Step 5 | −20 | 30 | 150 | R |
| Step 6 | −20 | 900 | 150 | H |
| Step 7 | −10 | 30 | 100 | R |
| Step 8 | −10 | 300 | 100 | H |
| Step 9 | −5 | 30 | 100 | R |
| Step 10 | −5 | 180 | 100 | H |
| Step 11 | 0 | 30 | 100 | R |
| Step 12 | 0 | 1440 | 100 | H |
| Step 13 | 5 | 30 | 100 | R |
| Step 14 | 5 | 60 | 100 | H |
| Step 15 | 15 | 120 | 100 | H |

Secondary Drying

| | | | | |
|---|---|---|---|---|
| Step 1 | 30 | 360 | 70 | H |

R: Ramp;
H: Hold
Total Lyophilization cycle Time: 112.83 Hrs

After lyophilization vacuum was broken with Nitrogen gas, the vials were stoppered, unloaded from lyophilizer, and sealed. Then the formulation was dried, off white to pale yellow colour cake with dark yellow colour at bottom of cake. At the corner of the vial dried collapsed/meltback cake. The lyophilized cake was easily reconstituted with water for injection within 3 minute forming clear yellow colour solution. No change in the pH/shift in pH of reconstituted solution was observed due to lyophilization and the results are shown in Table 46.

TABLE 146
Results of lyophilized cake of formulation_Batch No SF20000011

| | | | | pH | | Reconstituted | Osmolality |
|---|---|---|---|---|---|---|---|
| Batch | Appearance of | Reconstitution | Reconstitution | Before | After | Solution | (mOSmol/ |
| No. | Lyophilized Cake | Volume | Time | Lyo | Lyo | Appearance | Kg) |
| 011 | Dried, off white to Pale | 22.0 mL | 2 min | 4.7 | 4.59 | Clear | 741 |
| | Yellow color cake with | | | | | Yellow | |
| | dark yellow color at | | | | | color | |
| | bottom of cake. | | | | | Solution | |
| | At the corner of the | | | | | | |
| | vial dried | | | | | | |
| | collapse/meltback cake | | | | | | |

The as-prepared batch was loaded for stability study at 25° C.±2° C./60% RH±5% RH and 2°-8° C. up to 12 months. Placebo batch (Batch size: 400 mL) for respective formulation also manufactured and processed as per active batches.

TABLE 47
Stability data of formulations of compound of Formula
(I) formate salt for IV Injections (500 mg/Vial)

| | | pH 4.50 (L-Arginine) | | | | | | |
|---|---|---|---|---|---|---|---|---|
| | | Liquid Samples | | Lyophilized | 25° C. ± 2° C./60% | | | |
| Sr. | Test | Initial | Initial | Samples | RH ± 5% RH | | | |
| No. | Performed | Unfiltered | Filtered | Initial | 1 M | 2 M | 3 M | 6 M |
| 1 | Description- | — | — | # | ## | ### | ### | ### |
| | Lyo cake | | | | | | | |
| | Description- | ** | ** | ** | *** | ** | $ | $ |
| | Liquid | | | | | | | |
| 2 | Reconstitution | — | — | 7 min\hat{ } | 8 min\hat{ } | ~1 min | ~2 min | 8 min |
| | time | | | | | 30 sec | 58 sec | |
| 3 | pH | 4.6 | 4.7 | 4.59 | 4.53 | 4.73 | 4.59 | 4.52 |
| 4 | Water | — | — | 0.9 | 1.2 | 1.2 | 1.1 | 1.0 |
| | content (% | | | | | | | |
| | w/v) | | | | | | | |
| 5 | Osmolality | — | — | 741 | 754 | NA | 697 | NA |
| | (mOsmol/kg) | | | | | | | |
| 6 | Assay (% w/w) | 100.8 | 100.5 | 103.4 | 99.7 | 96.7 | 100.1 | 92.0 |
| 7 | Related | | | | | | | |
| | Substances | | | | | | | |
| | (%) | | | | | | | |
| | Highest | 0.41 | 0.41 | 0.29 | 1.97 | 2.7 | 3.622 | 5.512 |
| | Unknown | (0.82) | (0.82) | (1.26) | (0.82) | (1.25) | (1.26) | (1.24) |
| | (RRT) | | | | | | | |
| | Total | 1.38 | 1.66 | 1.32 | 2.97 | 4 | 4.3 | 6.1 |
| | Unknown | | | | | | | |
| | Total | 1.4 | 1.7 | 1.3 | 2.97 | 4 | 4.3 | 6.1 |
| | impurities | | | | | | | |
| 8 | Particulate | | | | | | | |
| | matter | | | | | | | |
| | Test^{¥} | | | | | | | |
| | ≥10μ | NA | NA | 1667 | NA | NA | 90774 | NA |
| | (NMT 6000 | | | | | | | |
| | particles/ | | | | | | | |
| | container) | | | | | | | |
| | ≥25μ | NA | NA | 29 | NA | NA | 513827 | NA |
| | (NMT 600 | | | | | | | |
| | particles/ | | | | | | | |
| | container) | | | | | | | |

| | | pH 4.50 (L-Arginine) | | | | | |
|---|---|---|---|---|---|---|---|
| Sr. | Test | 5° C. ± 3° C. | | | | | |
| No. | Performed | 1 M | 2 M | 3 M | 6 M | 9 M | 12 M |
| 1 | Description- | # | ### | ### | ### | ### | ### |
| | Lyo cake | | | | | | |
| | Description- | ** | ** | ** | ** | ** | ** |
| | Liquid | | | | | | |
| 2 | Reconstitution | 8 min\hat{ } | ~1 min | ~2 min | ~2 min | ~2 min | ~5 min |
| | time | | 30 sec | 55 sec | 55 sec | 46 sec | 20 sec |
| 3 | pH | 4.54 | 4.72 | 4.6 | 4.61 | 4.64 | 4.65 |
| 4 | Water | 0.9 | 1.2 | 1.0 | 1.3 | 1.4 | 1.5 |
| | content (% | | | | | | |
| | w/v) | | | | | | |
| 5 | Osmolality | 778 | NA | 693 | 732 | 752 | 752 |
| | (mOsmol/kg) | | | | | | |
| 6 | Assay (% w/w) | 100.2 | 99.5 | 104.9 | 101.7 | 102.2 | 103.1 |
| 7 | Related | | | | | | |
| | Substances | | | | | | |
| | (%) | | | | | | |
| | Highest | 0.29 | 0.45 | 0.247 | 0.951 | 0.399 | 0.460 |
| | Unknown | (1.26) | (1.25) | (0.82) | (1.24) | (0.87) | (1.27) |
| | (RRT) | | | | | | |
| | Total | 1.38 | 1.3 | 0.9 | 2.0 | 1.9 | 1.6 |
| | Unknown | | | | | | |
| | Total | 1.4 | 1.3 | 0.9 | 2.0 | 1.9 | 1.6 |
| | impurities | | | | | | |
| 8 | Particulate | | | | | | |
| | matter | | | | | | |
| | Test^{¥} | | | | | | |
| | ≥10μ | NA | NA | 1607 | 3153 | NA | NA |
| | (NMT 6000 | | | | | | |
| | particles/ | | | | | | |
| | container) | | | | | | |
| | ≥25μ | NA | NA | 38 | 97 | NA | NA |
| | (NMT 600 | | | | | | |
| | particles/ | | | | | | |
| | container) | | | | | | |

Sample 3M became suspension.
# Off-white to Pale Yellow colour shrinkage at bottom, Yellow colour layer at bottom of vial;
## Pale yellow to red colour cake with shrinkage at bottom;
** Clear yellow colour solution;
### Off white to red colour cake with shrinkage; yellow colour layer at bottom;
\hat{ }Time including 5 Min hold after addition of WFI;
$ Light Yellowish to orange coloured suspension;
~without 5 min hold;
^{¥}Particulate Matter Test: Done after dilution with 5D injection in 1:5 ratio.

From the above Table 47 it could be inferred that the colour of lyophilized cake was observed from yellow to reddish colour at 25° C.±2° C./60% RH±5% RH and at 2°-8° C. after 2 months. No change in pH of formulation was observed at both stability conditions. Decrease in assay and increase in impurities was observed at 25° C.±2° C./60% RH±5% RH. No significant difference was observed in assay and impurity profile at 2°-8° C. Based on observation of lyophilized cake of 500 mg/vial formulation of compound of Formula (I) formate salt for Injection, it was further evaluated at 250 mg/vial to improve the lyophilized cake structure to make it more elegant and pharmaceutically acceptable.

Formulation of Compound of Formula(I) Formate Salt for Injection 250 mg/Vial For Lyophilization Cycle Verification and Stability Study

TABLE 15
Formulation of Compound of formula (I) formate salt
for Injection 250 mg/vial (Batch Size: 900 mL)

| Sr. | | Quantity (mg/mL) | |
|---|---|---|---|
| No. | Ingredients | 129 | |
| 1 | Compound of Formula (I) formate | 20 | (2%) |
| | salt | | |
| 2 | Ascorbic Acid | 40 | (4%) |
| 3 | Lactic Acid | 18 | (1.8%) |
| 4 | Niacinamide | 25 | (2.5% |
| 5 | L-Arginine | 50 | (5%) |

| | | |
|---|---|---|
| 6 | Water For Injection (Qs to) | 1 mL |
| 7 | Target pH | 4.50 ± 0.1 |

Formulation as shown in Table 48 was prepared by the process as described below. Ascorbic acid was dissolved in water for injection (20% of batch size, 25° C.-30° C.) and was dissolved by stirring for 30 min followed by addition of lactic acid under stirring for 15 min at RT, and compound of Formula(I) formate salt under stirring for 150 min at RT to obtain the first solution. To the first solution, niacinamide was added and dissolved under stirring for 30 min. at RT to obtain a second solution. To the second solution added additional 40% of WFI (of total batch size) under stirring for 45 min and L-arginine (pH modifier) was added under stirring for 30 min at RT. WFI was added to makeup to the volume of solution to batch size. Then the solution was filtered through 0.2μ PES filter (47 mm, Lot No. 071715407, Sartorius) using vacuum filtration assembly to obtain the formulation. The 25.7 mL of filtered solution was filled in 50 mL clear USP type I Tubular vials, half stopper the vial and was loaded for lyophilization. The lyophilization cycle used for the formulation is as provided in Table 49 below.

TABLE 16
Recipe of the lyophilization cycle of formulation of
compound of Formula (I) formate salt in 250 mg/vial

| | | | | |
|---|---|---|---|---|
| | | Time | | R/H |
| Steps | Temp (° C.) | (min) | Vacuum (mT) | (ramp/hold) |

Freezing

| | | | | |
|---|---|---|---|---|
| Step 1 | 0 | 30 | — | R |
| Step 2 | 0 | 60 | — | H |
| Step 3 | −20 | 30 | — | R |
| Step 4 | −20 | 120 | — | H |
| Step 5 | −45 | 60 | — | R |
| Step 6 | −45 | 240 | — | H |
| Step 7 | −20 | 30 | — | R |
| Step 8 | −20 | 180 | — | H |
| Step 9 | −45 | 30 | — | R |
| Step 10 | −45 | 360 | — | H |

Extra Freezing

| | |
|---|---|
| Freeze | −45 |
| Extra Freeze | 30 |
| Condenser | −40 |
| Vacuum | 500 |

Primary Drying

| | | | | |
|---|---|---|---|---|
| Step 1 | −30 | 20 | 250 | R |
| Step 2 | −30 | 1200 | 250 | H |
| Step 3 | −25 | 30 | 200 | R |
| Step 4 | −25 | 600 | 200 | H |
| Step 5 | −20 | 30 | 150 | R |
| Step 6 | −20 | 600 | 150 | H |
| Step 7 | −10 | 30 | 100 | R |
| Step 8 | −10 | 300 | 100 | H |
| Step 9 | −5 | 30 | 100 | R |
| Step 10 | −5 | 120 | 100 | H |
| Step 11 | 0 | 30 | 100 | R |
| Step 12 | 0 | 600 | 100 | H |
| Step 13 | 5 | 30 | 100 | R |
| Step 14 | 5 | 120 | 100 | H |
| Step 15 | 15 | 30 | 100 | R |
| Step 16 | 15 | 120 | 100 | H |

Secondary Drying

| | | | | |
|---|---|---|---|---|
| Step 1 | 30 | 240 | 70 | H |

R: Ramp;
H: Hold
Total Lyophilization cycle Time: 88.33 Hrs

For solubilization compound of Formula (I) formate salt took longer time i.e. about 3 hrs. and some dust like particles were found to be visible. The unfiltered bulk solution was translucent yellow colour but after filtration from 0.2p filter it was found clear, yellow colour solution. There was no significant change in assay results of unfiltered and filtered bulk solution.

After lyophilization vacuum was broken with nitrogen gas and the vials were stoppered, unloaded from lyophilizer and sealed. Dried, intact, off white to pale yellow colour cake with dark yellow colour at bottom of cake and No Shrinkage or meltback of lyophilized cake was observed. At bottom of vial, yellow colour ring was observed, which could be due to phase separation of drug substance during lyophilization. The cake was reconstituted using 11.5 mL WFI, resulting solution was clear yellow colour solution. For reconstitution 11.5 mL WFI was added to vial then allowed to stand for 5 min at RT to ensure the proper wetting of solid then vigorously shaked for 2-3 minutes. Upper intact cake was got easily solubilized on addition of WFI without any shaking. The dried yellow layer of bottom requires vigorous shaking to get solubilized.

TABLE 50
Results of lyophilized cake of compound of Formula (I) formate salt in 250 mg/vial strength

| | Before | | | | | | |
|---|---|---|---|---|---|---|---|
| | Lyophilization | | | | | | |
| | Unfiltered | Filtered | After Lyophilization | | | | |
| | solution | solution | Appearance of | | | | Reconstituted |
| Batch | Assay | Assasy | Lyophilized | Reconstitution | Reconstitution | | Solution |
| No. | (%) | (%) | Cake | Volume | Time | pH | Appearance |
| SF20000129 | 101.4 | 101.9 | Dried, off | 11.5 mL | 2 min | 4.54 | Clear |
| | | | white to | | | | Yellow |
| | | | Pale Yellow | | | | color |
| | | | color cake | | | | Solution |
| | | | with dark | | | | |
| | | | yellow color | | | | |
| | | | at bottom of | | | | |
| | | | cake. | | | | |

Inference on Compound of Formula (I) Formate Salt Lyophilized Formulation in 250 mg/Vial Strength

Compound of Formula (I) formate salt lyophilized formulation in 250 mg/vial strength, lyophilization of formulation resulted in pharmaceutically acceptable lyophilized cake structure formation. The batch is loaded for stability study at 25° C.±2° C./60% RH±5% RH and 2°-8° C. up to 24 months. Placebo batch formulation also manufactured and processed as per active batch. The manufacturing process and its process component compatibility study was performed, and results obtained were satisfactory with all the process components. Hence it can be concluded the prepared formulation of the compound of Formula (I) formate salt for injection is compatible with the components such as Stainless steel 316L, USP Type 1 Glass, 0.2 PES filter, 0.2μ PVDF filter, PharmaPure AL242029 tubing, and Sani Tech 50© tubing upto 24 hrs.

It could be understood that the reconstituted formulation of compound of Formula (I) formate salt for Injection with 250 mg/vial should be used within 24 hrs after its reconstitution when stored at room temperature.

The reconstituted solution of compound of Formula (I) formate salt for Injection 250 mg/mL was further diluted with 5% Dextrose Injection IP and should be used within 10 hrs after its dilution when stored at room temperature. The concentrations of compound of Formula (I) formate salt such as 1.0 mg/mL and 3.33 mg/mL in diluted with 5% Dextrose Injection IP were recommended for its administration.

### Temperature Excursion Study and Freeze Thaw Study

Two different batches of the lyophilized formulation of the compound of Formula (I) formate salt and placebo was manufactured for evaluating Freeze thaw and temperature excursion stability. For Freeze-Thaw Cycling stability study, the exposure conditions were −20° C. for 24 Hrs. followed by 25° C.±2° C./60%±5% RH for 24 Hrs. This exposure was considered as completion of one Freeze thaw cycle. The formulation along with secondary pack was exposed to total 3 freeze thaw cycles. For lyophilized formulation i.e. compound of Formula (I) formate salt for IV Injection along with secondary pack was exposed for respective storage condition and respective duration. Exposure conditions were −20° C. for 24 Hrs and 48 Hrs and 40° C.±2° C./75%±5% RH for 24 Hrs. and 48 Hrs.

The formulation of the compound of Formula (I) formate salt drug product for Injection 250 mg/vial was found to be stable up to 24 hrs even when the formulation was exposed to different temperature conditions from −20° C. to 40° C.±2° C.

The compound of Formula (I) formate salt for Injection 250 mg/vial was also found to be stable up to 144 hrs (3 Cycle of Freeze thaw study duration) even if the formulation was exposed to different temperature conditions i.e. from −20° C.±2° C. to 25° C.±2° C.

### Photostability Study

The lyophilized formulation of the compound of Formula (I) formate salt was manufactured as described herein. Formulations were packed in various packages and was subjected to illumination to evaluate their photostabilities.

Lyophilised formulation (250 mg/vial) in 50 mL clear USP Type 1 glass vial was kept as such for light exposure till the overall required illumination was achieved. In another example, lyophilised formulation in 50 mL clear USP Type 1 glass vial was kept in secondary carton box and exposed to the light source till the overall required illumination was achieved. Similarly, the formulations were packed in aluminium foil, and carton box and were subjected to illumination. For comparative purpose control samples and placebos were also subjected to illumination. Table 51A shows the details of the packaging materials used. Similarly, only the active component i.e the compound of Formula (I) formate salt was subjected to photostability study and the details of the packaging materials are shown in Table 51B.

TABLE 51A
Details of Packaging material used:

| Sr. | | | |
|---|---|---|---|
| No. | Description | Manufacturer | Lot No. |
| 1 | 50 mL/20 mm clear/flint Tubular | Ompi (Stevanto Group) | B003240002 |
| | USP Type-1 glass vials | | |
| | (NEG Clear Glass Vials 50 mL 40 × | | |
| | 1.5 × 73 mm) | | |
| 2 | 20 mm Lyo Stopper | Datwyler Pharma Packaging | 31011357 |
| | (V9172 FM460/0 ISAF1 015 DARK | India Private Limited | |
| | GREY) | | |
| 3 | 20 mm Flip Top Royal Blue Grain | Shakai Packaging Private | SAM19380 |
| | Finish Aluminium Seal | Limited | |

TABLE 51B
Details of Packaging material used

| Sr. | | | |
|---|---|---|---|
| No. | Description | Manufacturer | Lot No. |
| 1 | 20 mL/20 mm amber color | Schott Kaisha | SKV1802725 |
| | tubular USP Type-1 | Pvt Ltd | |
| | glass vials | | |
| 2 | V9048 FM457/0 ISAF1 015 | Datwyler Pharma | 31132640 |
| | GREY | Packaging India | |
| | | Private Limited | |
| 3 | Secondary pack - E flute | Sneha Packaging | Not Applicable |
| | carton | | |
| 4 | Petri Plates | Not Applicable | Not Applicable |

Photostabilities of the formulations comprising the compound of Formula (I) formate salt for Injection and only compound of Formula (I) formate salt was evaluated as per ICH-Q1B guidelines.

The formulation of compound of Formula (I) formate salt for Injection 250 mg/vial was found to be insensitive to light. The remaining components in formulation were providing the light protection effect by forming solid cake structure above the API layer at the bottom of lyophilized cake.

The photostability study indicated that the compound of Formula (I) drug as a formate salt was found to be sensitive to light as discoloration was observed. Therefore, the compound of Formula (I) formate salt should be protected from light during manufacturing process of formulations. However, the formulation was found to be photoinsensitive. Thus, the formulation of the present disclosure was photostable and enhanced the stability of the compound of Formula (I) formate salt.

### Vmax Study

The objectives of a volume maximization study (Vmax) constant pressure study was to determine the sterilizing grade filters and membrane based prefilters capacity and provide sizing for sterilizing grade filters and membrane-based prefilters for a given unit operation.

Trial was conducted under manual operation using Virus Vmax vessel with filters connected to Virus Vmax vessel with help of luer-lock connection. Prior to the operation, filter was wetted/conditioned with water for injection and buffer. Moreover, before the process, devices were vented properly to avoid any air entrapment. Cumulative volume measurement as a function of time was recorded during the experiment, which in turn is used as a measure of the performance of the filter (At a constant pressure). t/V(time/volume) as a function of time (t) was plotted to draw a slope to calculate the capacity (Vmax) and initial volumetric flow (Ji) of the filter based on gradual pore plugging model. Minimum filtration area (Amin) required for batch volume (VB) in time (tB) was calculated using the following formula.

$$\frac{{A}_{min}}{{V}_{B}} = \frac{1}{{V}_{max}} + \frac{1}{{J}_{i} \times {t}_{B}}$$

In above equation V=VB=Process volume (L)

The filters evaluated: Sartoguard 1.2μ/0.2μ PES Nominal Filter as PREFILTER; followed by Sartopore 0.2μ PES absolute filter as ASEPTIC FILTER, as shown in Table 51C.

TABLE 51C

| Filter | Filter | Pore | | | Merck Part | Lot | Filter |
|---|---|---|---|---|---|---|---|
| Family | Name | size | | Material | Number | Number | Area |
| Milligard | Milligard | 1.2/0.45 | μm | PES | SAMP4A25NB6 | COAB89953 | 3.5 cm² |
| Millipore | Express | 0.2 | μm | PES | SGEPA25NB6 | COAB18271 | 3.5 cm² |
| | SHF | | | | | | |

The formulation of compound of formula (I) formate salt for Injection 250 mg/vial was found to be easily filterable. No filter clogging and flow decay was observed. The results (Table 51D) indicated that no adsorption of formulation of compound of Formula (I) formate salt drug product over the filter. No significant change in the related substances.

TABLE 51D
Results for Vₘₐₓ Study

| | | Compound of formula (I) | | |
|---|---|---|---|---|
| | | formulation_ B. No. SF20000203 | | |
| | | | Prefiltered | Aseptic |
| | | | Solution | filtered |
| Sr. | | Unfiltered | (1.2 μ/0.2 | solution |
| No. | Test Performed | solution | μ PES) | (0.2 μ PES) |
| 1 | Description-Liquid | # | ** | ** |
| 2 | pH | 4.53 | 4.55 | 4.54 |
| 3 | Assay (% w/v) | 99.6 | 100.4 | 101.5 |
| 4 | Related Substances (%) | | | |
| | Highest Unknown | 0.35 (0.82) | 0.30 (0.82) | 0.35 (0.82) |
| | (RRT) | | | |
| | Total impurities | 1.2 | 1.1 | 1.2 |

### Observations on the Formulations of the Present Disclosure

From the examples above on development of the formulation of the compound of Formula (I) formate salt, it could be understood that the drug product i.e the compound of Formula (I) formate salt was not stable in solution state for longer duration, hence it was necessary to prepare as a formulation and lyophilize the formulation for improved stability.

Further based on 6 months stability data of the prototype formulation batches, the formulation of compound of formula (I) formate salt for Injection was found to be stable at 2°-8° C. As the pH decreased from pH 4.50 to pH 3.50 impurity profile of the formulation also decreased at higher temperature i.e., at 40° C.±2° C./75% RH±5% RH and at 25° C.±2° C./60% RH±5% RH. Also, formulations with 1-arginine of 500 mg/vial and 250 mg/vial strength was charged for long term stability study. The formulations also comprises two acids combinations i.e., ascorbic acid and lactic acid. Further to reduce the acid concentration in formulation, 1-Arginine was added. The reduced strength from 500 mg/vial to 250 mg/vial, improved the lyophilized cake structure elegance with pharmaceutical acceptability and the lyophilized cake was produced using lyophilization cycle time period of 3-4 days as disclosed herein.

For use of the formulation in injection, the lyophilized formulation was reconstituted with reconstituting agent and a diluent. The reconstituting agent is water for injection and for dilution, 5% Dextrose (diluent) was used. The reconstituted formulation was found to be stable and compatible up to 10 hrs. after its dilution at room temperature. 1.0 mg/mL and 3.33 mg/mL diluted formulation can be used when dilution is required. With the formulation comprising 20 mg/mL of compound of Formula (I) formate salt drug product, the product strength was targeted at 250 mg/vial. Accordingly, the formulation of the present disclosure is provided in Table 52 below.

TABLE 52
Formulation of compound of formula (I) formate
salt drug product for Injection 250 mg/vial

| Sr. | | | |
|---|---|---|---|
| No. | Ingredients | % Weight (% w/v) | Quantity (mg/mL) |
| 1 | Compound of Formula | 2 | 20 |
| | (I) formate salt | | |
| 2 | Ascorbic acid | 4 | 40 |
| 3 | Lactic Acid | 1.8 | 18 |
| 4 | Niacinamide | 2.5 | 25 |
| 5 | L-Arginine | 5 | 50 |
| 6 | Water For Injection | Qs | Qs to 1 mL |
| 7 | Target pH | pH 4.50 ± 0.2 | pH 4.50 ± 0.2 |

### Kit Comprising the Formulation.

The formulation of the present disclosure could be used in the form of a kit wherein the kit comprised a first compartment and a second compartment. The first compartment comprised the as-prepared formulation. In another example, the kit comprised the reconstituted formulation or a lyophilized formulation. The second compartment comprised water. The kit is optionally comprised of an accessory which is selected from diluent bag, infusion tubing, needles, or connector.

### Method of Treating Bacterial Infections

### Dose Finding Study of Formulations in a Neutropenic Murine Model of Thigh Infection Caused by A. Baumannii ATCC 17978

The objective of this study was to investigate the efficacy of the formulations comprising the compound of Formula(I) formate salt as disclosed herein, across the dose range of 10 mg/kg to 120 mg/kg administered by subcutaneous (SC) injection every 8 hours (q8h) using a 26 hour neutropenic murine dual thigh infection model by infecting with A. baumannii ATCC 17978. The well-established antibiotics meropenem and polymyxin B were used as a comparator controls.

Mice were infected intramuscularly with A. baumannii ATCC 17978 two hours prior to treatment with Compound of Formula(I) formate salt administered subcutaneously (SC) at 10, 30, 60, 90, and 120 mg/kg q8h and comparator controls meropenem (administered SC at 200 mg/kg q8h) and polymyxin B (administered SC at 25 mg/kg q8h). Animals were euthanized at 2-hour post infection (pre treatment controls), or 26-hours post-infection (vehicle control and all treated groups. Data was obtained from all animals at early timepoints.

Compound of Formula (I) formate salt lyophilized vials as formulation were reconstituted with SWFI and reconstituted solution was diluted with SWFI to the required dose strength for dosing through subcutaneous route. Similarly, for dosing control group animals the lyophilized placebo vials were reconstituted with SWFI and diluted to required strength using SWFI.

When the SC dose range of 10 to 120 mg/kg q8h was evaluated, Compound of Formula (I) formate salt was effective at reducing the A. baumannii ATCC 17978 bacterial burden at 10 and 30 mg/kg q8h, demonstrated either a bacteriostatic or killing effect against A. baumannii ATCC 17978 at a dose of 60 mg/kg q8h and logarithmic killing was achieved at higher doses of 90 and 120 mg/kg q8h in the neutropenic mouse thigh model.

Compound of Formula (I) formate salt was more effective than polymyxin B (administered SC at 25 mg/kg q8h) at doses ≥90 mg/kg q8h and Subcutaneous meropenem (200 mg/kg q8h) against this A. baumannii strain. FIG. 4 depicts the CFU/g of mouse thighs infected with Acinetobacter baumannii ATCC 17978 and treated subcutaneously with [Compound of Formula(I) formate salt] or meropenem (200 mg/kg) or polymyxin B (25 mg/kg) administered q8h (every 8 hours). A neutropenic murine dual thigh model of A. baumannii ATCC 17978 was used to demonstrate the efficacy of compound of Formula (I) formate salt when administered subcutaneously in vivo. When the SC dose range of 10 to 120 mg/kg q8h was evaluated across three replicate studies, compound of Formula (I) formate salt was effective at reducing the A. baumannii ATCC 17978 bacterial burden at 10 and 30 mg/kg q8h and caused logarithmic killing at ≥60 mg/kg q8h in a general dose-dependent manner.

Thus, the formulation comprising the compound of Formula (I) formate salt demonstrated either a bacteriostatic or killing effect against A. baumannii ATCC 17978 at a dose of 60 mg/kg q8h with logarithmic killing achieved at higher doses of 90 and 120 mg/kg q8h in the neutropenic mouse thigh model. The formulation of the present disclosure was more effective than polymyxin B (administered SC at mg/kg q8h) at doses ≥90 mg/kg q8h and subcutaneous meropenem (200 mg/kg q8h) against this A. baumannii strain.

### Advantages of the Present Disclosure

The present disclosure provides a formulation comprising the compound of Formula (I) formate salt with a solubilizing agent, a pH modifier and a hydrotropic agent. The formulation of the present disclosure is stable for a long duration of time and is stable at a pH range of 2 to 6. The formulation of the present disclosure is stable up to a temperature in a range of −25° C. to 10° C. Also, the formulation of the present disclosure can be lyophilized and stability can be improved. The present disclosure also provides a reconstituted formulation which makes the formulation in an injectable form. The present disclosure further provides a process for preparing the formulations which are easy to adapt and suitable for large scale production. The present disclosure also provides a formulation which is capable of treating infections caused by microorganisms. The present disclosure also provides a method of treating or preventing a disease or condition mediated by the microorganism's and the disease or condition is selected from complicated and uncomplicated urinary tract infections (cUTI, eg., pyelonephritis, cystitis), intra-abdominal infections (cIAI), bloodstream infections, Hospital-Acquired Bacterial Pneumonia (HABP, Nosocomial Pneumonia), Ventilator-Associated Bacterial Pneumonia (VABP), Community-Acquired Bacterial Pneumonia (CABP), cystic fibrosis secondary infections (CFI), skin and soft tissue infections (SSTIs), endocarditis, meningitis, dysentery and diarrhoae, typhoid, Clostridium difficile associated colitis and diarrhoea (CDAD), Helicobacter pylori associated peptic ulcer, sexually transmitted diseases caused by Chlamydia trachomatis, gonorrhoea caused by Neisseria gonorrhoeae, syphilis caused by Treponema pallidum, or bioterrorism associated bacterial diseases caused by Anthrax (Bacillus anthracis), Bubonic Plague (Yersinia pestis), Tularemia (Francisella tularensis), Glanders (Burkholderia mallei), Melioidosis (Burkholderia pseudomallei) and Q fever (Coxiella burnetiid).

1. A formulation comprising:
a. a compound of Formula (I) or its salt thereof; 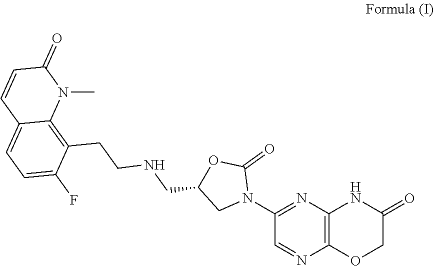 [SMILES: Cn1c(=O)ccc2ccc(F)c(CCNC[C@H]3CN(c4cnc5c(n4)NC(=O)CO5)C(=O)O3)c21]
b. a solubilising agent;
c. a hydrotropic agent; and
d. a pH modifier.

2. The formulation as claimed in claim 1, wherein the formulation comprises the compound of Formula (I) or its salt thereof in a weight range of 1 to 30% (w/w); the solubilising agent in a weight range of 5 to 55% (w/w); the hydrotropic agent in a weight range of 1 to 30% (w/w); and the pH modifier in a weight range of 0.5 to 30% (w/w).

3. The formulation as claimed in claim 1, wherein the solubilising agent is selected from lactic acid, ascorbic acid, acetic acid, propionic acid, succinic acid, gluconic acid, benzoic acid, tartaric acid, glutaric acid, malic acid, fumaric acid or combinations thereof, the pH modifier is selected from potassium hydroxide, sodium hydroxide, l-arginine, histidine, glycine, sodium bicarbonate, or combinations thereof, the hydrotropic agent is selected from niacinamide, sodium benzoate, sodium citrate sodium acetate, or combinations thereof; and the salt of compound of Formula (I) is selected from formate, mesylate, esylate, besylate, tosylate, actetate, propionate, fumarate, maleate, tartarate, succinate, glycolate, glutamate, aspartate, hydrochloride, hydrobromide, or sulphate.

4. The formulation as claimed in claim 1, wherein the formulation is stable at a temperature in a range of −70° C. to 10° C.; and the formulation has a pH in a range of 2 to 6.

5. (canceled)

6. The formulation as claimed in claim 1, wherein the formulation comprises a vehicle; and the vehicle is water.

7. The formulation as claimed in claim 1, wherein the formulation is capable of killing or inhibiting the growth of microorganisms; and the microorganism is selected from bacteria, virus, fungi, and protozoa.

8. The formulation as claimed in claim 1, wherein the formulation is an anti-infective agent against microorganism; and wherein the microorganism is selected from Escherichia coli, Pseudomonas aeruginosa, Klebsiella pneumoniae, Acinetobacter baumannii, Enterobacter cloacae, Citrobacter spp., Proteus spp., Serratia marcescens, Salmonella species, Morganella morganii, Klebsiella oxytoca, Klebsiella aerogenes, Providencia spp., Neisseria gonorrhoea, Mycoplasma spp., Campylobacter sp, Fusobacterium spp., Bacteroides fragilis, Prevotella sp., Shigella sp., Helicobacter pylori, Ureaplasma spp., Burkholderia gladioi, Burkholderia multivorans, Pandorea apista, Burkholderia cepacia, Burkholderia pseudomallei, Burkholderia mallei, Stenotrophomonas maltophilia, Achromobacter sp., Ralstonia picketii, Legionella pneumophila, Clostridium difficile, Staphylococcus aureus, Coagulase-negatives Staphylococcus, Streptococcus pyogenes, Streptococcus pneumoniae, Enterococcus faecium, Enterococcus faecalis, Bacillus anthracis, Yersinia pestis and Francisella tularensis.

9. (canceled)

10. The formulation as claimed in claim 1, wherein the formulation inhibits an enzyme selected from bacterial gyrase, topoisomerase IV, or combinations thereof.

11. The formulation as claimed in claim 1, wherein the formulation further comprises an additive, a therapeutic agent, or combinations thereof; the additive is selected from sorbitol, dextrose, sodium phosphate monobasic, sodium phosphate dibasic, sodium citrate, sodium bicarbonate, sodium chloride, or combinations thereof.

12. (canceled)

13. A lyophilized formulation comprising: the formulation as claimed in claim 1.

14. A reconstituted formulation comprising: the lyophilized formulation as claimed in claim 13, with a reconstituting agent and a diluent, wherein the reconstituting agent is water; the diluent is selected from dextrose, sorbitol, sodium bicarbonate, glucose, mannitol, sucrose, or sodium chloride; and the diluent is in a weight range of 0.9-25% (w/v) in a first solvent; and the first solvent is water.

15. (canceled)

16. (canceled)

17. A kit comprising:
a. a first compartment comprising the formulation as claimed in claim 1;
b. a second compartment comprising a facilitating agent; and
c. optionally an accessory,
wherein the facilitating agent is selected from water for injection; and the accessory is selected from diluent bag, infusion tubing, needles and connector.

18. (canceled)

19. A process for preparing the formulation as claimed in claim 1, the process comprising:
a. contacting a solubilising agent with a compound of Formula (I) or it's salt thereof in the presence of a second solvent under stirring at a temperature in a range of 18 to 40° C. to obtain a first solution;
b. adding a hydrotropic agent to the first solution under stirring to obtain a second solution; and
c. mixing a pH modifier to the second solution optionally followed by addition of a vehicle to obtain the formulation.

20. The process as claimed in claim 19, wherein two or more solubilising agents are mixed prior to contacting with the compound of Formula (I) or its salt thereof.

21. The process as claimed in claim 19, wherein the first solution has a pH in a range of 2 to 3; the second solution has a pH in a range of 2.5 to 4; the second solvent is water; and the vehicle is water.

22. The process as claimed in claim 19, wherein the formulation is filtered after adding the vehicle.

23. (canceled)

24. A process for preparing the lyophilized formulation as claimed in claim 13, the process comprising: freezing a formulation comprising a compound of Formula (I) or its salts thereof, a solubilising agent, a hydrotropic agent, and a pH modifier at a temperature in a range of 0 to −50° C. followed by drying at a pressure in a range of 10 pbar to 1 bar, to obtain the lyophilized formulation.

25. (canceled)

26. A process for preparing the reconstituted formulation as claimed in claim 14, the process comprising: contacting a lyophilized formulation comprising a compound of Formula (I) or its salts thereof, a solubilising agent, a hydrotropic agent, and a pH modifier with a diluent followed by addition of a reconstituting agent.

27. (canceled)

28. The process as claimed in claim 26, wherein the reconstituted formulation is stable for a time period range of 20 to 30 hours at a temperature in a range of 20 to 35° C.: the reconstituted formulation has a pH in a range of 3 to 5; and has osmolality in a range of 300-500 mOs-mol/kg.

29.-31. (canceled)

32. A method of treating a microbial infection, the method comprising administering an effective amount of the formulation as claimed in claim 1 to a subject in need thereof, wherein the microbial infection is caused by bacteria, virus, fungi, or protozoa.

33. (canceled)

34. A method of treating or preventing a disease or a condition, the method comprising administering an effective amount of the formulation as claimed in claim 1 to a subject in need thereof.

35. The method as claimed in claim 34, wherein the disease or the condition is mediated by gram-positive, gram-negative bacterial species or combinations thereof; and the disease or the condition is selected from complicated and uncomplicated urinary tract infections (cUTI, eg., pvelonephritis, cystitis), intra-abdominal infections (cIAI), bloodstream infections, Hospital-Acquired Bacterial Pneumonia (HABP, Nosocomial Pneumonia), Ventilator-Associated Bacterial Pneumonia (VABP), Community-Acquired Bacterial Pneumonia (CABP), cystic fibrosis secondary infections (CFI), skin and soft tissue infections (SSTIs), endocarditis, meningitis, dysentery and diarrhoae, typhoid, Clostridium difficile associated colitis and diarrhoea (CDAD), Helicobacter pylori associated peptic ulcer, sexually transmitted diseases caused by Chlamydia trachomatis, gonorrhoea caused by Neisseria gonorrhoeae, syphilis caused by Treponema pallidum, or bioterrorism associated bacterial diseases caused by Anthrax (Bacillus anthracis), Bubonic Plague (Yersinia pestis), Tularemia (Francisella tularensis), Glanders (Burkholderia mallei), Melioidosis (Burkholderia pseudomallei) or Q fever (Coxiella burnetii).

36. (canceled)
